(19) United States
(12) Patent Application Publication
(10) Pub. No.: US 2022/0081395 A1
(43) Pub. Date: Mar. 17, 2022

(54) DISULPHIDE COMPOUNDS

(71) Applicant: NEEM BIOTECH LIMITED, ABERTILLERY GWENT (GB)

(72) Inventors: Christopher Gregory Newton, Abertillery (GB); Gareth James Streeet EVANS, Abertiviery (GB); Kodande Ranganatha Nagraja RAO, Abertillery (GB); Shasita Sheroze KHOKHAR, Abertillery (GB); Philip John DUDFIELD, Abertillery (GB)

(73) Assignee: NEEM BIOTECH LIMITED, ABERTILLERY GWENT (GB)

(21) Appl. No.: 17/426,281

(22) PCT Filed: Jan. 22, 2020
(86) PCT No.: PCT/GB2020/050137

§ 371 Date: Jul. 28, 2021

(30) Foreign Application Priority Data
Jan. 30, 2019 (GB) 1901237.6

(51) Int. Cl. C07C 323/65; C07D 261/08; C07D 239/26; C07D 307/38; A61P 31/04

(52) U.S. Cl. CPC C07C 323/65; C07D 261/08; A61P 31/04; C07D 307/38; C07D 239/26

(57) ABSTRACT

Disulphide compounds of Formula (I) where Y is sulphuryl or sulphinyl Z is phenyl or substituted phenyl and the other variables are as defined in the claims; pharmaceutical compositions comprising these compounds and methods for making these compounds. These compounds are useful in therapeutic methods for treating microbial infection, inflammation and reducing the formation of blood clots and have non-therapeutic use as antimicrobial agents, anti-inflammatory agents and as anti-thrombotic agents.

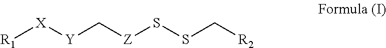

### TECHNICAL FIELD

The present invention generally relates to disulphide compounds of formula (I) and pharmaceutical compositions comprising one or more disulphide compounds of formula (I). The present invention also relates to methods of making the disulphide compounds of formula (I). The present invention further relates to the various uses of the disulphide compounds of formula (I), for example as an antimicrobial agent, as an anti-inflammatory agent, as an anti-thrombotic agent, for the treatment of a wound, and/or for the treatment of cystic fibrosis.

### BACKGROUND

A number of disulphide compounds are known to have physiological effects. For example, ajoene, a disulphide compound found in garlic extract, is known to have antimicrobial, anti-inflammatory, and anti-thrombotic effects. It is therefore desirable to obtain new alternative or improved disulphide compounds that may have one or more advantageous physiological effects.

### SUMMARY

In accordance with a first aspect of the present invention there is provided a compound of formula (I),

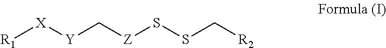

wherein,

R₁ is phenyl, substituted phenyl, cycloalkyl, substituted cycloalkyl, heterocyclyl, substituted heterocyclyl, or alkyl;

X is (CH₂)ₙ;

n is 0 to 10;

Y is sulphuryl or sulphinyl;

Z is phenyl or substituted phenyl;

R₂ is hydrogen, phenyl, substituted phenyl, alkyl, alkenyl, heterocyclyl, substituted heterocyclyl, —CH(CO₂R₃)(NHCO₂R₄), —CH(CO₂H)(NH₂), or —CH(CO₂R₅)(R₆); and

R₃, R₄, R₅, and R₆ are alkyl;

or a pharmaceutically acceptable salt, ester, or prodrug thereof.

In certain embodiments, the compound of formula (I) is according to formula (IA) or formula (IB),

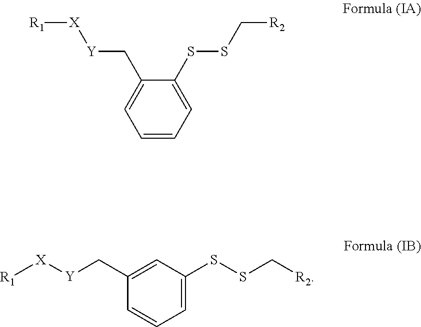

In accordance with a second aspect of the present invention there is provided a pharmaceutical composition comprising a compound, pharmaceutically acceptable salt, ester, or prodrug of the first aspect of the present invention and a pharmaceutically acceptable excipient and/or carrier and/or diluent.

In accordance with a third aspect of the present invention there is provided a compound, pharmaceutically acceptable salt, ester, prodrug, or pharmaceutical composition of any aspect of the present invention, including all embodiments thereof, for use in a therapeutic method for treating a microbial infection and/or for treating inflammation and/or for reducing the formation of blood clots and/or for treating a wound and/or for treating cystic fibrosis.

In accordance with a fourth aspect of the present invention there is provided a use of a compound, pharmaceutically acceptable salt, ester, prodrug, or pharmaceutical composition of any aspect of the present invention, including all embodiments thereof, in the manufacture of a medicament for treating a microbial infection and/or for treating inflammation and/or for reducing the formation of blood clots and/or for treating a wound and/or for treating cystic fibrosis.

In accordance with a fifth aspect of the present invention there is provided a therapeutic method for treating a microbial infection and/or for treating inflammation and/or for reducing the formation of blood clots and/or for treating a wound and/or for treating cystic fibrosis, wherein the method comprises administering a compound, pharmaceutically acceptable salt, ester, prodrug, or pharmaceutical composition of any aspect of the present invention, including all embodiments thereof, to a subject.

In accordance with a sixth aspect of the present invention there is provided a non-therapeutic use of a compound, pharmaceutically acceptable salt, ester, prodrug, or pharmaceutical composition of any aspect of the present invention, including all embodiments thereof, as an antimicrobial agent and/or as an anti-inflammatory agent and/or as an anti-thrombotic agent. For example, there is provided herein an in vitro use of a compound, pharmaceutically acceptable salt, ester, prodrug, or pharmaceutical composition of any aspect of the present invention, including all embodiments thereof, as an antimicrobial agent and/or as an anti-inflammatory agent and/or as an anti-thrombotic agent.

In accordance with a seventh aspect of the present invention there is provided a method for making a compound, pharmaceutically acceptable salt, ester, or prodrug, of the first aspect of the present invention. The method may, for example, proceed via steps 1 to 4 or via steps 1 to 5 of reaction scheme (IA) or reaction scheme (IB):

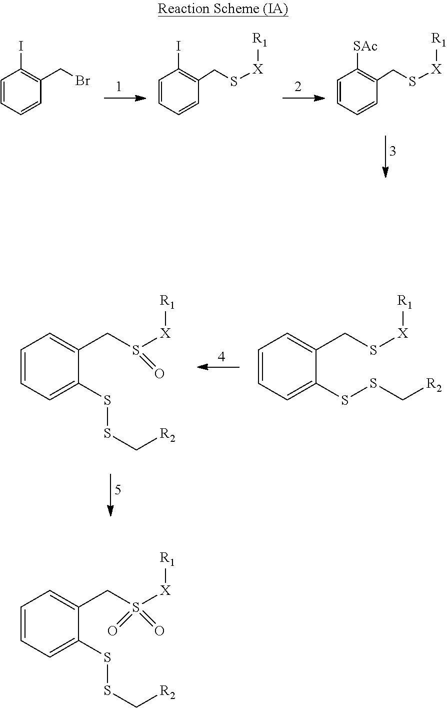

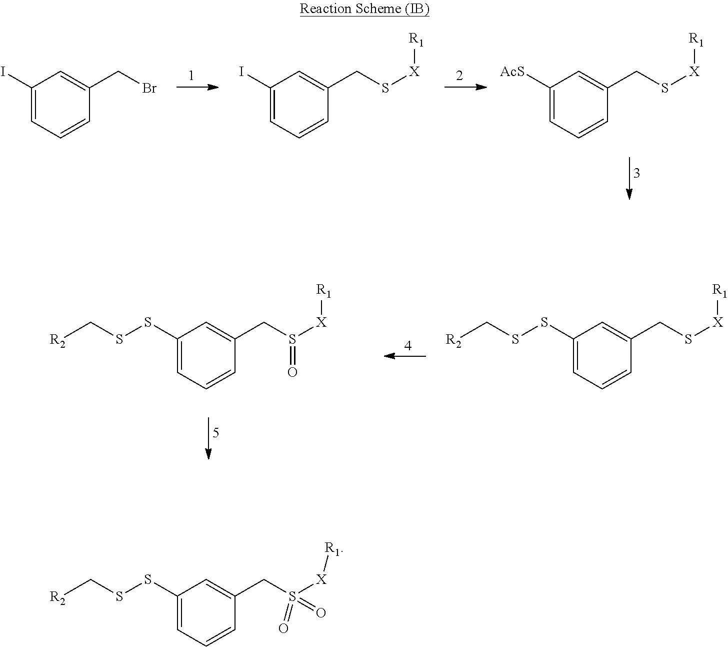

The starting material in Reaction Scheme (IA) and/or Reaction Scheme (IB) may, for example, have one or more additional substituents if Z is substituted phenyl in the compound according to formula (I).

In certain embodiments of any aspect of the present invention, the following compound is excluded from the definition of the compound of formula (I),

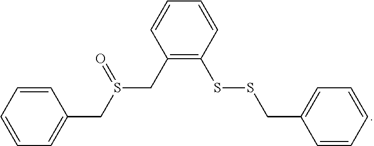 [SMILES: O=S(Cc1ccccc1)Cc1ccccc1SSCc1ccccc1]

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

The present invention may be described with reference to the following non-limiting Figures in which:

FIG. 1 shows the plot of concentration versus % P. aeruginosa biofilm mass for compound A compared to that of ajoene, cysteamine, HDMF, gallium nitrate, and C-30;

FIG. 2 shows the plot of concentration versus % S. aureus biofilm mass for compound A compared to that of ajoene, cysteamine, HDMF, gallium nitrate, and C-30;

FIG. 3 shows the plot of concentration of compound B versus % P. aeruginosa biofilm respiration (as a % of control).

FIG. 4 shows the plot of % scratch remaining in a monolayer of HaCaT cells over time when the HaCaT cells are exposed to S. aureus exudate pre-treated with test compound compared to HaCaT cells not exposed to any bacterial exudate or exposed to S. aureus pre-treated with vehicle.

FIG. 5 shows the plot of % scratch remaining in a monolayer of HaCaT cells over time when the HaCaT cells are exposed to P. aeruginosa exudate pre-treated with test compound compared to HaCaT cells not exposed to any bacterial exudate or exposed to P. aeruginosa pre-treated with vehicle.

### DETAILED DESCRIPTION

Compounds and Pharmaceutical Compositions

The present invention is based, at least in part, on the surprising finding that compounds of formula (I) have an advantageous antimicrobial activity. For example, embodiments of the present invention are based on the surprising finding that compounds of formula (I) provide an improved antimicrobial activity in comparison to other disulphide compounds.

Hereinafter, the invention shall be described according to preferred embodiments of the present invention and by referring to the accompanying description. However, it is to be understood that limiting the description to the preferred embodiments of the invention is merely to facilitate discussion of the present invention and it is envisioned that those skilled in the art may devise various modifications without departing from the scope of the appended claims.

The terms generally used hereinbefore and hereinafter have for preference the meanings indicated below, unless indicated otherwise, whereby more specific meanings may be used independently of one another in preferred embodiments of the present invention instead of the general definitions, these more specific significances describing especially preferred embodiments of the invention.

Where the term “at least one” or “one or more” occurs hereinbefore and hereinafter, this signifies for example one to ten, for preference one to three, and in particular one or, further, two of the features enumerated, such as components. Where ranges are indicated, such as weight percentage ranges, these include the limit values indicated; thus, for example, “between a and b” signifies “from and including a up to and including b”.

The compounds of formula (I) are defined as follows:

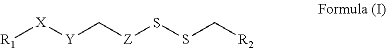

wherein,

R₁ is phenyl, substituted phenyl, cycloalkyl, substituted cycloalkyl, heterocyclyl, substituted heterocyclyl, or alkyl;

X is (CH₂)ₙ;

n is 0 to 10;

Y is sulphuryl or sulphinyl;

Z is phenyl or substituted phenyl;

R₂ is hydrogen, phenyl, substituted phenyl, alkyl, alkenyl, heterocyclyl, substituted heterocyclyl, —CH(CO₂R₃)(NHCO₂R₄), —CH(CO₂H)(NH₂), or —CH(CO₂R₅)(R₆); and

R₃, R₄, R₅, and R₆ are alkyl.

In certain embodiments, the compound of formula (I) is a compound according to formula (IA) or a compound according to formula (IB),

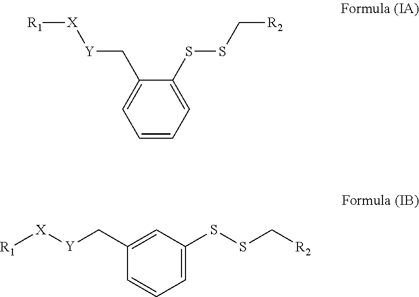

wherein R₁, R₂, X, and Y are the same as defined in relation to Formula (I).

In certain embodiments, the definition of formula (I) or formula (IA) or formula (IB) excludes the compound

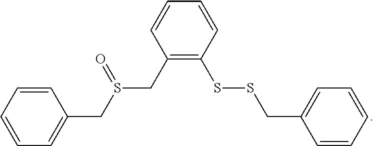 [SMILES: O=S(Cc1ccccc1)Cc1ccccc1SSCc1ccccc1]

The term “phenyl” as used herein refers to a radical that is derived from benzene by the removal of one or more hydrogen atom(s). For example, when R₁ and/or R₂ is phenyl, the radical has the formula C₆H₅ and is attached to the adjacent atom in the compound of formula (I) where the hydrogen atom has been removed. For example, when Z is phenyl, the radical has the formula C₆H₄ and is attached to the adjacent atoms in the compound of formula (I) where the hydrogen atoms have been removed.

The term “substituted phenyl” as used herein refers to a radical that is derived from benzene by the removal of one or more hydrogen atoms, wherein one or more of the remaining hydrogen atoms on the benzene ring is replaced by a functional group. The substituted phenyl may, for example, comprise one, two, three, four, or five functional groups. The substituted phenyl may, for example, comprise one or two functional groups. The substituted phenyl may, for example, comprise only one functional group.

The substitution(s) may, for example, occur at the meta-, ortho-, or para-position on the phenyl ring. For example, the substitution(s) may occur at the para-position on the phenyl ring.

The term “cycloalkyl” used herein refers to a radical derived from a saturated cyclic hydrocarbon by removal of a hydrogen atom. The cycloalkyl may, for example, comprise from 3 to 12 carbon atoms or from 3 to 10 carbon atoms or from 3 to 8 carbon atoms or from 3 to 6 carbon atoms.

The term “substituted cycloalkyl” used herein refers to a radical derived from a saturated cyclic hydrocarbon by removal of a hydrogen atom, wherein one or more of the remaining hydrogen atoms on the cyclic hydrocarbon is replaced by a functional group. The substituted cycloalkyl may, for example, comprise one, two, three, four, or five functional groups. The substituted cycloalkyl may, for example, comprise one or two functional groups. The substituted cycloalkyl may, for example, comprise only one functional group. The substituted cycloalkyl may, for example, comprise from 3 to 12 carbon atoms or from 3 to 10 carbon atoms or from 3 to 8 carbon atoms or from 3 to 6 carbon atoms.

The term “heterocyclyl” used herein refers to a radical derived from a saturated or unsaturated cyclic structure that has atoms of at least two different elements as members of the ring by removal of a hydrogen atom. The heterocyclyl may, for example, have one or more carbon atoms and one or more atoms selected from nitrogen, oxygen, and sulphur, as members of the ring. The heterocyclyl may, for example, be a heteroaryl. The heterocyclyl may, for example, be isoxazole, furan, thiophene or pyrimidine. The heterocyclyl may, for example, be isoxazole, furan, or pyrimidine. For example, the heterocyclyl may be furan.

The term “substituted heterocyclyl” used herein refers to a radical derived from a saturated or unsaturated cyclic structure that has atoms of at least two different elements as members of the ring, wherein one or more of the remaining hydrogen atoms on the heterocyclyl is replaced by a functional group. The substituted heterocyclyl may, for example, be a substituted heteroaryl. The substituted heterocyclyl may, for example, comprise one, two, three, four, or five functional groups. The substituted heterocyclyl may, for example, comprise one or two functional groups. The substituted heterocyclyl may, for example, comprise only one functional group. Each functional group may, for example, be an alkyl group (e.g. methyl) or an alkoxy group (e.g. methoxy). The substitution may, for example, occur on a carbon atom. The heterocyclyl may, for example, include an oxazole ring, an isoxazole ring, a furan ring, a thiophene ring or a pyrimidine ring. The heterocyclyl may, for example, include an oxazole ring, an isoxazole ring, a furan ring or a pyrimidine ring. The substituted heterocyclyl may, for example, be a substituted isoxazole ring or a substituted pyrimidine ring.

Each functional group may, for example, independently be selected from an alkyl group, a haloalkyl group, an ester group, an alkoxy group, a halogen group, a sulphonyl group (e.g. an alkylsulphonyl) group, a haloalkoxy group, or an amine group. Thus, the substituted phenyl may, for example, be an alkylphenyl, a haloalkylphenyl, an alkylbenzoate, an alkoxyphenyl, a halophenyl, an alkylphenyl sulphone, a haloalkoxyphenyl, or an aminophenyl. Each functional group may, for example, independently be selected from an alkyl group, an alkoxy group, a halogen group, a sulphonyl group (e.g. an alkylsulphonyl group), or a haloalkyl group.

The term “sulphuryl” used herein refers to a group having the formula —S(═O)₂.

The term “sulphinyl” used herein refers to a group having the formula —S(═O)—.

The term “alkyl” used herein refers to a radical derived from a saturated linear or branched hydrocarbon by removal of a hydrogen atom. The alkyl may, for example, comprise from 1 to 8 carbon atoms or from 1 to 4 carbon atoms or from 1 to 2 carbon atoms.

The term “alkenyl” as used herein refers to a radical derived from an unsaturated straight or branched chain hydrocarbon. The alkenyl group may, for example, contain 1, 2, or 3 carbon-carbon double bonds. For example, the alkenyl group may contain 1 carbon-carbon double bond. The alkenyl may, for example, comprise from 2 to 8 carbon atoms or from 2 to 4 carbon atoms or from 2 to 3 carbon atoms. The alkenyl may, for example, be ethenyl.

The term “haloalkyl” used herein refers to a radical derived from a saturated linear or branched hydrocarbon by removal of a hydrogen atom, where one or more of the remaining hydrogen atoms of the alkyl group is replaced by a halogen. The haloalkyl may, for example, comprise from 1 to 8 carbon atoms or from 1 to 4 carbon atoms or from 1 to 2 carbon atoms. The haloalkyl may, for example, comprise from 1 to 8 halogen atoms or from 1 to 5 halogen atoms or from 1 to 4 halogen atoms or from 1 to 3 halogen atoms. The halogen atoms may each, independently, be selected from iodine, chlorine, bromine, and fluorine. The haloalkyl may, for example, be a halomethyl. For example, the haloalkyl may be a trihalomethyl. For example, the haloalkyl may be trifluoromethyl. Thus, the substituted phenyl may be haloalkylphenyl for example halomethylphenyl. The haloalkylphenyl may, for example, be trihalomethylphenyl. The haloalkylphenyl may, for example, be trifluoromethyl phenyl. The trifluoromethyl group may, for example, occur at the para-position of the phenyl ring.

The term “ester” used herein refers to a group having the formula —C(═O)OR, wherein R is an alkyl group. The alkyl is a saturated linear or branched chain hydrocarbon. The alkyl may, for example, comprise from 1 to 8 carbon atoms or from 1 to 4 carbon atoms or from 1 to 2 carbon atoms.

The term “alkoxy” used herein refers to a group having the formula —OR, wherein R is an alkyl group. The alkyl is a saturated linear or branched chain hydrocarbon. The alkyl may, for example, comprise from 1 to 8 carbon atoms or from 1 to 4 carbon atoms or from 1 to 2 carbon atoms. The alkyl may, for example, contain 1 carbon atom (a methyl group) such that the alkoxyphenyl is methoxyphenyl. The alkoxy may, for example, occur at the para-position of a phenyl ring.

The term “haloalkoxy” used herein refers to a group having the formula —OR, wherein R is an alkyl group, wherein one or more of the hydrogen atoms of the alkyl group is replaced by a halogen. The haloalkoxy may, for example, comprise from 1 to 8 carbon atoms or from 1 to 4 carbon atoms or from 1 to 2 carbon atoms. The haloalkoxy may, for example, comprise from 1 to 8 halogen atoms or from 1 to 5 halogen atoms or from 1 to 4 halogen atoms or from 1 to 3 halogen atoms. The halogen atoms may each, independently, be selected from iodine, chlorine, bromine, and fluorine.

The term “halo” or “halogen” used herein refers to any of fluorine, chlorine, bromine, iodine, and astatine. In certain embodiments, the halogen is selected from fluorine, chlorine, bromine, and iodine. In certain embodiments, the halogen is selected from fluorine, chlorine, and bromine. In certain embodiments, the halogen is fluorine or chlorine. In certain embodiments, the halogen is fluorine. A substituted phenyl may be fluorophenyl. The halogen may, for example, occur at the para-position of a phenyl ring.

The term “sulphonyl” used herein refers to a group having the formula —S(O)(O)R where R is a functional group, for example selected from hydrogen, halogen, alkyl, haloalkyl, alkoxy, haloalkoxy, carbonyl (—C(O)R″ where R″ is an alkyl group), or ester. For example, R may be selected from halogen and alkyl, for example alkyl.

The term “alkylsulphonyl” used herein refers to a group having the formula —S(O)(O)R, wherein R is an alkyl group. The alkyl group may, for example, comprise from 1 to 8 carbon atoms or from 1 to 4 carbon atoms or from 1 to 2 carbon atoms. The alkyl may, for example, contain 1 carbon atom (a methyl group) such that the alkylsulphone is methylsulphone (thus the substituted phenyl would be methyl phenyl sulphone). The alkylsulphone may, for example, occur at the para-position of a phenyl ring.

The term “amine” used herein refers to a group having the formula NRR', wherein R and R′ are each independently selected from hydrogen, halogen, alkyl, haloalkyl, alkoxy, haloalkoxy, carbonyl (—C(O)R″ where R″ is an alkyl group), ester, or alkylsulphonyl.

R₁ is phenyl, substituted phenyl, cycloalkyl, substituted cycloalkyl, heterocyclyl, substituted heterocyclyl, or alkyl. R₁ may, for example, be phenyl, substituted phenyl, heterocyclyl or alkyl. R₁ may, for example, be phenyl, heterocyclyl or alkyl. R₁ may, for example, be phenyl, substituted phenyl, or alkyl. R₁ may, for example, be phenyl or substituted phenyl. R₁ may, for example, be phenyl.

X is (CH₂)ₙ, where n is 0 to 10. n may, for example, be 0 to 8 or 0 to 5 or 0 to 4 or 0 to 3 or 0 to 2. n may, for example, be 1. n may, for example, be 0.

Y is sulphuryl or sulphinyl. Y may, for example, be sulphuryl. Y may, for example, be sulphinyl.

Z is phenyl or substituted phenyl. Z may, for example, be phenyl.

R₂ is hydrogen, phenyl, substituted phenyl, alkyl, alkenyl, heterocyclyl, substituted heterocyclyl, —CH(CO₂R₃)(NHCO₂R₄), —CH(CO₂H)(NH₂), or —CH(CO₂R₅)(R₆). R₂ may, for example, be hydrogen, phenyl, substituted phenyl, alkenyl, heterocyclyl, substituted heterocyclyl, —CH(CO₂R₃)(NHCO₂R₄), —CH(CO₂H)(NH₂), or —CH(CO₂R₅)(R₆). R₂ may, for example, be hydrogen, phenyl, substituted phenyl, alkyl, alkenyl, heterocyclyl, or substituted heterocyclyl. R₂ may, for example, be hydrogen, phenyl, substituted phenyl, alkenyl, heterocyclyl, or substituted heterocyclyl.

The —CH(CO₂R₃)(NHCO₂R₄), —CH(CO₂H)(NH₂), and —CH(CO₂R₅)(R₆) groups may, for example, be in (R) or (S) configuration. The —CH(CO₂R₃)(NHCO₂R₄), —CH(CO₂H)(NH₂), and —CH(CO₂R₅)(R₆) groups may, for example, be in (R) configuration.

The R₃, R₄, R₅, and R₆ groups, when present, may, for example, each independently comprise from 1 to 8 carbon atoms or from 1 to 6 carbon atoms or from 1 to 4 carbons. For example, the R₃ and R₄ groups when present may each independently comprise from 3 to 8 carbon atoms or from 3 to 6 carbon atoms or from 3 to 4 carbon atoms. For example, the R₂ and R₄ groups when present may be a secondary or tertiary alkyl group. For example, the R₃ and R₄ groups when present may each independently be a butyl group, for example a tertiary butyl group. For example, the R₅ and R₆ groups when present may each independently comprise from 1 to 4 carbon atoms or from 1 to 3 carbon atoms or from 1 to 2 carbon atoms. For example, the R₅ and R₆ groups when present may be a methyl group.

The —CH(CO₂R₃)(NHCO₂R₄) group may, for example, be —CH(CO₂t-Bu)(NHCO₂ t-Bu).

The —CH(CO₂R₅)(R₆) group may, for example, be —CH(CO₂Me)(Me).

When R₂ is substituted phenyl, the phenyl may be substituted with one or more functional groups selected from alkoxy, halogen, sulphonyl (e.g. alkylsulphonyl), and haloalkyl. For example, when R₂ is substituted phenyl, the phenyl may be substituted with one functional group selected from alkoxy, halogen, sulphonyl (e.g. alkylsulphonyl), and haloalkyl. For example, when R₂ is substituted phenyl, the phenyl may be substituted at the para position with one functional group selected from alkoxy, halogen, sulphonyl (e.g. alkylsulphonyl), and haloalkyl. The alkoxy functional group may, for example, comprise from 1 to 8 carbon atoms, for example from 1 to 4 carbon atoms or from 1 to 2 carbon atoms. For example, the alkoxy functional group may be methoxy. The halogen functional group may, for example, be fluorine, chlorine, or bromine. For example, the halogen functional group may be fluorine. The sulphonyl functional group may, for example, be an alkylsulphonyl functional group. The alkyl in the alkylsulphonyl functional group may, for example, comprise from 1 to 8 carbon atoms, for example from 1 to 4 carbon atoms or from 1 to 2 carbon atoms. For example, the sulphonyl functional group may be a methylsulphonyl functional group. The haloalkyl group may, for example, comprise from 1 to 8 carbon atoms, for example from 1 to 4 carbon atoms or from 1 to 2 carbon atoms. The haloalkyl group may, for example, comprise from 1 to 4 halogen atoms. The haloalkyl group may, for example, be a trihaloalkyl group. For example, the haloalkyl group may be a trihalomethyl group such as trifluoromethyl.

When R₂ is alkyl, the alkyl may, for example comprise from 1 to 8 carbon atoms or from 1 to 4 carbon atoms or from 1 to 2 carbon atoms. The alkyl may, for example, be a linear alkyl or a branched chain alkyl. The alkyl may, for example, be a secondary or tertiary alkyl. The alkyl may, for example, be —CH(CH₂CH₃)(CH₃) or —CH(CH₃)(CH₃).

When R₂ is alkenyl, the alkenyl may, for example, contain 1, 2, or 3 carbon-carbon double bonds. For example, when R₂ is alkenyl, the alkenyl group may contain 1 carbon-carbon double bond. When R₂ is alkenyl, the alkenyl may comprise from 2 to 8 carbon atoms or from 2 to 4 carbon atoms or from 2 to 3 carbon atoms. When R₂ is alkenyl, the alkenyl may, for example, be ethenyl.

When R₂ is heterocylyl or substituted heterocyclyl, the heterocyclic ring may be isoxazole, furan, or pyrimidine, for example isoxazole or pyrimidine. The isoxazole or pyrimidine ring may, for example, be substituted with one or more functional groups. For example, the isoxazole or pyrimidine ring may be substituted with one or two functional groups. Each functional group may, for example, each be an alkyl group (e.g. methyl) or an alkoxy group (e.g. methoxy). The functional group(s) may, for example, each (e.g. both) be present on a carbon atom in the isoxazole or pyrimidine ring. For example, the functional group(s) may be present on the carbon atoms numbered 3 and/or 5 on an isozazole ring (where the oxygen ring atom is numbered 1 and the nitrogen ring atom is numbered 2). For example, the functional group(s) may be present on the carbon atoms numbered 4 and/or 6 on a pyrimidine ring (where one nitrogen ring atom is numbered 1 and the other is numbered 3). For example, the heterocyclyl may be

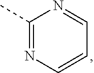

where the dotted line indicates the position where the heterocyclyl is bonded to the compound of formula (I). For example, the substituted heterocyclyl may be

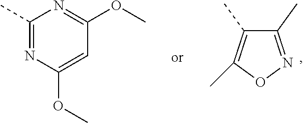

where the dotted lines indicate the position where the substituted heterocyclyl is bonded to the compound of formula (I).

When R₁ is substituted phenyl, the phenyl may be substituted with one or more functional groups selected from alkoxy, halogen, sulphonyl (e.g. alkylsulphonyl), and haloalkyl. For example, when R₁ is substituted phenyl, the phenyl may be substituted with one or more halogen groups, which may, for example, be fluorine. For example, when R₁ is substituted phenyl, the phenyl may be substituted with one fluorine atom, for example at the para-position.

The compound of formula (I) may, for example, be a compound of formula (IA) or formula (IB) wherein R₁ is phenyl. The compound of formula (I) may, for example, be a compound of formula (IA) or formula (IB) wherein R₁ is phenyl and Y is sulphuryl. The compound of formula (I) may, for example, be a compound of formula (IA) or formula (IB) wherein R₁ is phenyl and Y is sulphinyl. In certain embodiments, n is 1.

The compound of formula (I) may, for example, be a compound of formula (IA) or formula (IB) wherein R₁ is phenyl or heterocyclyl. The compound of formula (I) may, for example, be a compound of formula (IA) or formula (IB) wherein R₁ is phenyl or heterocyclyl and Y is sulphuryl. The compound of formula (I) may, for example, be a compound of formula (IA) or formula (I B) wherein R₁ is phenyl or heterocyclyl and Y is sulphinyl. In certain embodiments, n is 1. In certain embodiments, the heterocyclyl is a furan ring.

The compound of formula (I) may, for example, be a compound of formula (IA) or formula (IB) wherein R₁ is phenyl and R₂ is hydrogen, phenyl, substituted phenyl, alkyl, heterocyclyl, substituted heterocyclyl, —CH(CO₂R₃)(NHCO₂R₄), —CH(CO₂H)(NH₂), or —CH(CO₂R₅)(R₆). The compound of formula (I) may, for example, be a compound of formula (IA) or formula (IB) wherein R₁ is phenyl and R₂ is hydrogen, phenyl, alkyl, heterocyclyl, —CH(CO₂R₃)(NHCO₂R₄), —CH(CO₂H)(NH₂), or —CH(CO₂R₅)(R₆), phenyl substituted with alkoxy, halogen, sulphonyl (e.g. alkylsulphonyl), or haloalkyl, or heterocyclyl substituted with alkyl or alkoxy. In certain embodiments, n is 1.

The compound of formula (I) may, for example, be a compound of formula (IA) or formula (IB) wherein R₁ is phenyl or heterocyclyl and R₂ is hydrogen, phenyl, substituted phenyl, alkyl, heterocyclyl, substituted heterocyclyl, —CH(CO₂R₃)(NHCO₂R₄), —CH(CO₂H)(NH₂), or —CH(CO₂R₅)(R₆). The compound of formula (I) may, for example, be a compound of formula (IA) or formula (IB) wherein R₁ is phenyl or heterocyclyl and R₂ is hydrogen, phenyl, alkyl, heterocyclyl, —CH(CO₂R₃)(NHCO₂R₄), —CH(CO₂H)(NH₂), or —CH(CO₂R₅)(R₆), phenyl substituted with alkoxy, halogen, sulphonyl (e.g. alkylsulphonyl), or haloalkyl, or heterocyclyl substituted with alkyl or alkoxy. In certain embodiments, n is 1. In certain embodiments, the heterocyclyl is a furan ring.

The compound of formula (I) may, for example, be a compound of formula (IA) or formula (IB) wherein Y is sulphuryl and R₂ is hydrogen, phenyl, substituted phenyl, alkyl, heterocyclyl, substituted heterocyclyl, —CH(CO₂R₃)(NHCO₂R₄), —CH(CO₂H)(NH₂), or —CH(CO₂R₅)(R₆). The compound of formula (I) may, for example, be a compound of formula (IA) or formula (IB) wherein Y is sulphuryl and R₂ is hydrogen, phenyl, alkyl, heterocyclyl, —CH(CO₂R₃)(NHCO₂R₄), —CH(CO₂H)(NH₂), or —CH(CO₂R₅)(R₆), phenyl substituted with alkoxy, halogen, sulphonyl (e.g. alkylsulphonyl), or haloalkyl, or heterocyclyl substituted with alkyl or alkoxy. The compound of formula (I) may, for example, be a compound of formula (IA) or formula (IB) wherein Y is sulphinyl and R₂ is hydrogen, phenyl, substituted phenyl, alkyl, heterocyclyl, substituted heterocyclyl, —CH(CO₂R₃)(NHCO₂R₄), —CH(CO₂H)(NH₂), or —CH(CO₂R₅)(R₆). The compound of formula (I) may, for example, be a compound of formula (IA) or formula (IB) wherein Y is sulphinyl and R₂ is hydrogen, phenyl, alkyl, heterocyclyl, —CH(CO₂R₃)(NHCO₂R₄), —CH(CO₂H)(NH₂), or —CH(CO₂R₅)(R₆), phenyl substituted with alkoxy, halogen, sulphonyl (e.g. alkylsulphonyl), or haloalkyl, or heterocyclyl substituted with alkyl or alkoxy. In certain embodiments, n is 1.

The compound of formula (I) may, for example, be a compound of formula (IA) or formula (IB) wherein Ri is phenyl, Y is sulphuryl or sulphinyl, and R₂ is hydrogen, phenyl, substituted phenyl, alkyl, heterocyclyl, substituted heterocyclyl, —CH(CO₂R₃)(NHCO₂R₄), —CH(CO₂H)(NH₂), or —CH(CO₂R₅)(R₆). The compound of formula (I) may, for example, be a compound of formula (IA) or formula (IB) wherein R₁ is phenyl, Y is sulphuryl or sulphinyl, and R₂ is hydrogen, phenyl, alkyl, heterocyclyl, CH(CO₂R₃)(NHCO₂R₄), —CH(CO₂H)(NH₂), or —CH(CO₂R₅)(R₆), phenyl substituted with alkoxy, halogen, sulphonyl (e.g. alkylsulphonyl), or haloalkyl, or heterocyclyl substituted with alkyl or alkoxy. In certain embodiments, n is 1.

The compound of formula (I) may, for example, be a compound of formula (IA) or formula (IB) wherein R₁ is phenyl or heterocyclyl, Y is sulphuryl or sulphinyl, and R₂ is hydrogen, phenyl, substituted phenyl, alkyl, heterocyclyl, substituted heterocyclyl, —CH(CO₂R₃)(NHCO₂R₄), —CH(CO₂H)(NH₂), or —CH(CO₂R₅)(R₆). The compound of formula (I) may, for example, be a compound of formula (IA) or formula (IB) wherein R₁ is phenyl or heterocyclyl, Y is sulphuryl or sulphinyl, and R₂ is hydrogen, phenyl, alkyl, heterocyclyl, —CH(CO₂R₃)(NHCO₂R₄), —CH(CO₂H)(NH₂), or —CH(CO₂R₅)(R₆), phenyl substituted with alkoxy, halogen, sulphonyl (e.g. alkylsulphonyl), or haloalkyl, or heterocyclyl substituted with alkyl or alkoxy. In certain embodiments, n is 1. In certain embodiments, the heterocyclyl is a furan ring.

The compound of formula (I) may, for example, be a compound of formula (IA) or formula (IB) wherein R₁ is phenyl or substituted phenyl, n is 0 or 1, Y is sulphuryl or sulphinyl, Z is phenyl, and R₂ is hydrogen, alkyl, or alkenyl. The compound of formula (I) may, for example, be a compound of formula (IA) or formula (IB) wherein R₁ is phenyl, n is 1, Y is sulphuryl, Z is phenyl, and R₂ is hydrogen, alkyl, or alkenyl. The compound of formula (I) may, for example, be a compound of formula (IA) or formula (IB) wherein R₁ is phenyl, n is 1, Y is sulphuryl, Z is phenyl, and R₂ is hydrogen, or alkenyl.

The compound of formula (I) may, for example, be a compound of formula (IA) or formula (IB) wherein R₁ is phenyl or substituted phenyl or heterocyclyl, n is 0 or 1, Y is sulphuryl or sulphinyl, Z is phenyl, and R₂ is hydrogen, alkyl, or alkenyl. The compound of formula (I) may, for example, be a compound of formula (IA) or formula (IB) wherein R₁ is phenyl or heterocyclyl, n is 1, Y is sulphuryl, Z is phenyl, and R₂ is hydrogen, alkyl, or alkenyl. The compound of formula (I) may, for example, be a compound of formula (IA) or formula (IB) wherein R₁ is phenyl or heterocyclyl, n is 1, Y is sulphuryl, Z is phenyl, and R₂ is hydrogen, or alkenyl. In certain embodiments, the heterocyclyl is a furan ring.

In certain embodiments, the compound of formula (I) is one of the following compounds.

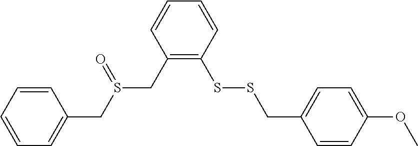 [SMILES: COc1ccc(CSSc2ccccc2CS(=O)Cc2ccccc2)cc1]

### 1-({[2-(benzyldisulfanyl)phenyl]methanesulfinyl}methyl)-4-methoxybenzene

δH (400 MHz, CDCl3): 3.79 (s, 3H, OCH3); 3.89 (s, 2H, CH2S═O); 3.93 (d, J.=12.8 Hz, 1H, CH2-S═O); 4.29 (d, J.=13.2 Hz, 1H, CH2-S═O), 6.78-7.71 (m, 13H, CH—Ar).

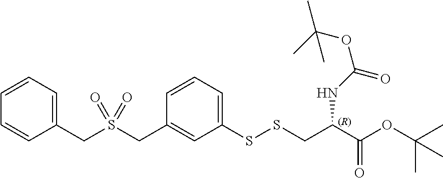

### tert-butyl-(2R)-2-{[(tert-butoxy)carbonyl]amino}-3-{[3-(phenylmethanesulfonylmethyl)phenyl]disulfanyl}propanoate

δH (400 MHz, CDCl3) 7.51 (1H, br s) overlapping 7.50 (1H, br s), 7.39 (5H, br s) overlapping 7.34 (1H, t, J=7.9 Hz), 7.26 (1H, br s), 5.27 (1H, br d, J=8.3 Hz), 4.43 (1H, ABX), 4.14 (2H, br s) overlapping 4.10 (2H, br s), 3.25 (1H, ABX), 3.13 (1H, ABX), 1.44 (9H, s) overlapping 1.41 (9H, s)

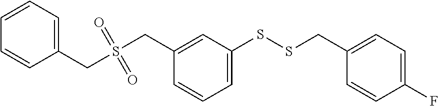 [SMILES: O=S(=O)(Cc1ccccc1)Cc1cccc(SSCc2ccc(F)cc2)c1]

### 1-fluoro-4-({[3-(phenylmethanesulfonylmethyl)phenyl]disulfanyl}methyl)benzene

δH (400 MHz, CDCl3) 7.42-7.32 (4H, m), 7.31-7.17 (6H, m), 7.09 (1H, br d, J=7.83 Hz), 6.93-6.86 (2H, m), 3.91 (2H, s), 3.89 (2H, m), 3.82 (1H, AB, J=13.0Hz), 3.75 (1H, AB, J=13.0 Hz)

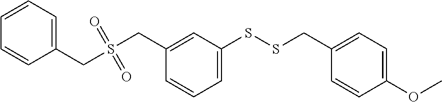 [SMILES: COc1ccc(CSSc2cccc(CS(=O)(=O)Cc3ccccc3)c2)cc1]

### 1-methoxy-4-({[3-(phenylmethanesulfonylmethyl)phenyl]disulfanyl}methyl)benzene

δH (400 MHz, CDCl3) 7.43-7.34 (7H, m), 7.27 (1H, t, J=7.7 Hz), 7.20-7.25 (3H, m), 6.78-6.73 (2H, m), 4.11 (2H, s), 4.02 (2H, s), 3.90 (2H, s), 3.74 (3H, s)

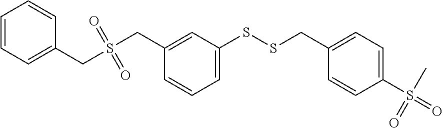 [SMILES: CS(=O)(=O)c1ccc(CSSc2cccc(CS(=O)(=O)Cc3ccccc3)c2)cc1]

### 1-methanesulfonyl-4-({[3-(phenylmethanesulfonylmethyl)phenyl]disulfanyl}methypbenzene

δH (400 MHz, CDCl3) 7.77-7.74 (2H, m), 7.45-7.40 (7H, m), 7.35-7.29 (2H, m), 7.21 (1H, t, J=7.7 Hz), 7.10 (1H, dm, J=7.6 Hz), 4.20 (2H, s), 4.00 (2H, s), 3.96 (2H, s), 2.99 (3H, s)

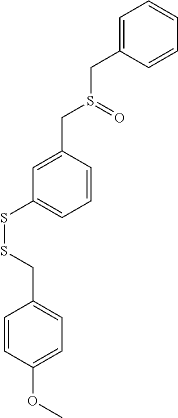 [SMILES: COc1ccc(CSSc2cccc(CS(=O)Cc3ccccc3)c2)cc1]

### 1-methoxy-4-({[3-(phenylmethanesulfinylmethyl)phenyl]disulfanyl}methyl)benzene

δH (400 MHz, CDCl3) 7.41-7.24 (8H, m), 7.19-7.14 (2H, m), 7.09 (1H, br d, J=7.6 Hz), 6.78-6.74 (2H, m), 3.94-3.77 (6H, overlapping multiplets), 3.74 (3H, s)

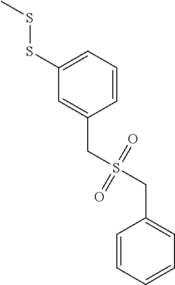 [SMILES: CSSc1cccc(CS(=O)(=O)Cc2ccccc2)c1]

### 1-(methyldisulfanyI)-3-(phenylmethanesulfonylmethyl)benzene

δH (400 MHz, CDCl3) 7.56-7.49 (2H, m), 7.44-7.36 (5H, m) overlapping 7.35 (1H, t, J=7.8 Hz), 7.23 (1H, dm) overlapping residual CHCI3, 4.14 (2H, s), 4.09 (2H, s), 2.43 (3H, s)

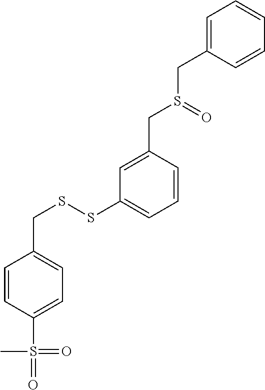 [SMILES: CS(=O)(=O)c1ccc(CSSc2cccc(CS(=O)Cc3ccccc3)c2)cc1]

### 1-methanesulfonyl-4-({[3-(phenylmethanesulfinylmethyl)phenyl]disulfanyl}methyl)benzene

δH (400 MHz, CDCl3) 7.78-7.74 (2H, m), 7.44-7.25 (9H, m), 7.21 (1H, t, J=7.7 Hz), 7.06 (1H, dm, J=7.6Hz), 3.9 (4H, s), 3.82 (1H, AB, J=13.0 Hz), 3.69 (1H, AB, J=13.0 Hz), 2.99 (3H, s)

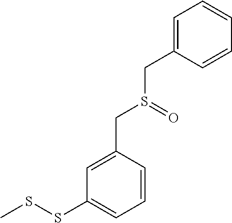 [SMILES: CSSc1cccc(CS(=O)Cc2ccccc2)c1]

### 1-(methyldisulfanyI)-3-(phenylmethanesulfinylmethyl)benzene

δH (400 MHz, CDCl3) 7.49 (1H, dm, J=7.9Hz), 7.45-7.27 (7H, m), 7.15 (1H, dm, J=7.6 Hz), 3.92 (2H, d, J=1.6 Hz) overlapping 3.90 (1H, AB, J=13.1 Hz), 3.82 (1H, AB, J=13.0 Hz), 2.44 (3H, s)

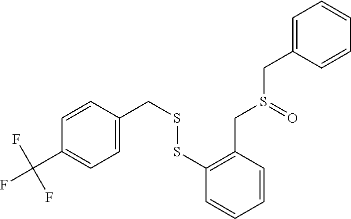 [SMILES: O=S(Cc1ccccc1)Cc1ccccc1SSCc1ccc(C(F)(F)F)cc1]

### 1-({[2-(phenylmethanesulfinylmethyl)phenyl]disulfanyl}methyl)-4-(trifluoromethyl)benzene

δH (300 Hz, CDCl3): 3.89 (d, J.=12.9 Hz, 1H, CH2-S═O); 3.93 (s, 2H, CH2-S═O), 4.01 (d, J.=3 Hz, 2H, CH2-S—S), 4.21 (d, J.=12.9 Hz, 1H, CH2-S—═O); 7.25-7.84 (m, 13H, CH—Ar).

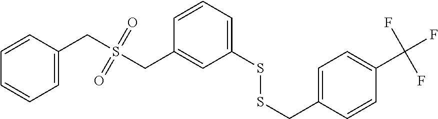 [SMILES: O=S(=O)(Cc1ccccc1)Cc1cccc(SSCc2ccc(C(F)(F)F)cc2)c1]

### 1-({[3-(phenylmethanesulfonylmethyl)phenyl]disulfanyl}methyl)-4-(trifluoromethyl)benzene

δH (400 MHz, CDCl3) 7.47-7.31 (11H, m), 7.23 (1H, t, J=7.7 Hz) overlapping residual CHCl3, 7.13 (1H, doublet of broad triplets, J=7.6 Hz), 4.14 (2H, s), 4.00 (2H, s) 3.94 (2H, s)

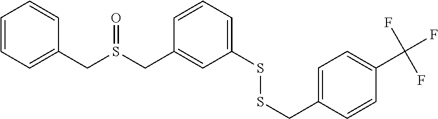 [SMILES: O=S(Cc1ccccc1)Cc1cccc(SSCc2ccc(C(F)(F)F)cc2)c1]

### 1-({[3-(phenylmethanesulfinylmethyl)phenyl]disulfanyl}methyl)-4-(trifluoromethyl)benzene

δH (400 MHz, CDCl3) 7.45 (2H, br. d, J=8.1 Hz), 7.42-7.27 (9H, m), 7.22 (1H, t, J=7.7 Hz), 7.07 (1H, doublet of broad triplets, J=7.6 Hz), 3.94 (2H, s), 3.92 (2H, s), 3.82 (1H, AB, J=13.0 Hz), 3.72 (1H, AB, J=13.0 Hz)

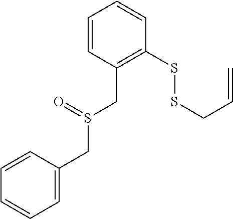 [SMILES: C=CCSSc1ccccc1CS(=O)Cc1ccccc1]

### 1-(phenylmethanesulfinylmethyl)-2-(prop-2-en-1-yldisulfanyl)benzene

δH (400 Hz, CDCl3): 3.37 (d, J.=7.2 Hz, 2H, CH2-S—S), 3.98 (d, J.=12.8 Hz, 1H, CH2-S═O); 4.04 (s, 2H, CH2-S═O); 4.36 (d, J.=12.8 Hz, 1H, CH2-S═O), 5.13-5.18 (m, 2H, CH2=), 5.76-5.82 (m, 1H, CH═), 7.34-7.81 (m, 9H, CH—Ar).

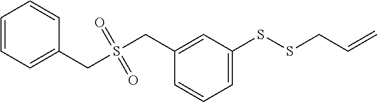 [SMILES: C=CCSSc1cccc(CS(=O)(=O)Cc2ccccc2)c1]

### 1-(phenylmethanesulfonylmethyl)-3-(prop-2-en-1-yldisulfanyl)benzene

δH (400 MHz, CDCl3) 7.56-7.51 (2H, m), 7.45-7.37 (5H, m), 7.34 (1H, td, J=7.6, 0.7 Hz), 7.23 (1H, dm, J=7.6 Hz), 5.81 (1H, ddt, J=17.0, 10.0, 7.4 Hz), 5.18-5.10 (2H, overlapping multiplets), 4.15 (2H, s), 4.10 (2H, s), 3.37 (2H, dt, J=7.4, 0.9 Hz)

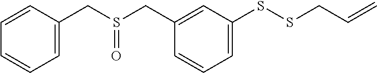 [SMILES: C=CCSSc1cccc(CS(=O)Cc2ccccc2)c1]

### 1-(phenylmethanesulfinylmethyl)-3-(prop-2-en-1-yldisulfanyl)benzene

δH (400 MHz, CDCl3) 7.48 (1H, dm, J=7.9 Hz), 7.43-7.26 (7H, m), 7.13 (1H, dm, J=7.7 Hz), 5.79 (1H, ddt, J=17.0, 9.9, 7.4 Hz), 5.16-5.08 (2H, overlapping multiplets), 3.91 (2H, d, J=2.7 Hz) overlapping 3.89 (1H, AB), 3.81 (1H, AB, J=13.0 Hz), 3.35 (2H, dm, J=7.1 Hz)

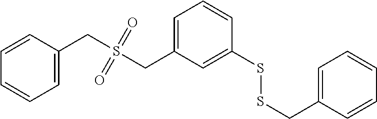 [SMILES: O=S(=O)(Cc1ccccc1)Cc1cccc(SSCc2ccccc2)c1]

### 1-(benzyldisulfanyl)-3-(phenylmethanesulfonylmethyl)benzene

δH (400 MHz, CDCl3) 7.43-7.33 (7H, m), 7.30-7.16 (7H, m) overlapping CHCl3 signal, 4.10 (2H, s), 4.02 (2H, s), 3.93 (2H, s)

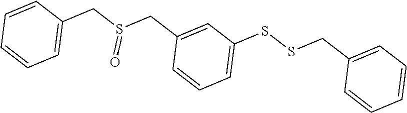

### 1-(benzyldisulfanyl)-3-(phenylmethanesulfinylmethyl)benzene

δH (400 MHz, CDCl3) 7.39-7.34 (3H, m), 7.30-7.16 (10H, m) over lapping CHCl3 signal, 7.09 (1H, dm, J=7.7 Hz), 3.92 (2H, s), 3.88 (2H, d, J=2.8z), 3.82 (1H, AB, J=13.0 Hz), 3.76 (1H, AB, J=13.0 Hz)

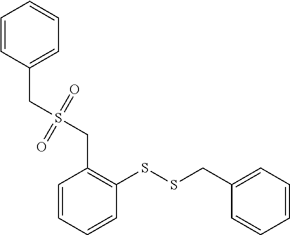 [SMILES: O=S(=O)(Cc1ccccc1)Cc1ccccc1SSCc1ccccc1]

### 1-(benzyldisulfanyl)-2-(phenylmethanesulfonylmethyl)benzene

δH (400 MHz, CDCl3) 7.68 (1H, dd, J=7.8, 1.2 Hz), 7.41-7.28 (7H, m), 7.26 (1H, dd, J=7.4, 1.4 Hz), 7.23-7.15 (5H, m), 4.36, (2H, s), 4.16 (2H, s) 3.91 (2H, s)

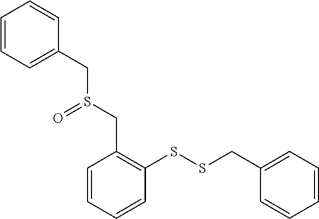 [SMILES: O=S(Cc1ccccc1)Cc1ccccc1SSCc1ccccc1]

### 1-(benzyldisulfanyl)-2-(phenylmethanesulfinylmethyl)benzene

δH (400 MHz, CDCl3) 7.63 (1H, dm, J=7.6 Hz), 7.40-7.18 (13H, m) overlapping CHCl3 signal, 4.23 (1H, AB, J=12.9 Hz), 3.96 (2H, s), 3.88 (2H, s) overlapping 3.86 (1H, AB)

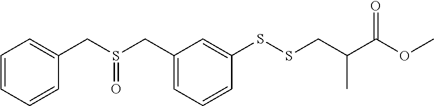 [SMILES: COC(=O)C(C)CSSc1cccc(CS(=O)Cc2ccccc2)c1]

### methyl 2-methyl-3-{[3-(phenylmethanesulfinylmethyl)phenyl]disulfanyl}propanoate

δH (400 MHz, CDCl3) 7.48 (1H, br d, J=8.0 Hz), 7.42-7.26 (7H, m), 7.14 (1H, br d, J=7.6 Hz), 3.95, 3.92 (1H, AB, J=13.0 Hz), 3.91, 3.87 (1H, AB, J=13.0 Hz), 3.88 (1H, AB, J=13.1 Hz), 3.81 (1H, AB, J=13.1 Hz), 3.65 (3H, s), 3.05 (1H, dd, J=13.3, 7.3 Hz), 2.84 (1H, hextet, J=7.0Hz), 2.71 (1H, dd, J=13.3, 6.6 Hz), 1.22 (3H, d, J=7.0 Hz)

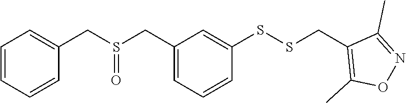 [SMILES: Cc1noc(C)c1CSSc1cccc(CS(=O)Cc2ccccc2)c1]

### 3,5-dimethyl-4-({[3-(phenylmethanesulfinylmethyl)phenyl]disulfanyl}methyl)-1,2-oxazole

δH (400 MHz, CDCl3) 7.41-7.32 (4H, m), 7.32-7.26 (4H, m), 7.11 (1H, dm, J=7.6 Hz), 3.96, 3.93 (1H, AB, J=13.5 Hz) overlapping 3.93, 3.89 (1H, AB, J=13.6 Hz), 3.86 (1H, AB, J=13.0 Hz), 3.76 (1H, AB, J=13.0 Hz), 3.66 (2H, s), 2.23 (3H, s), 2.18 (3H, s)

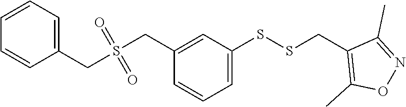 [SMILES: Cc1noc(C)c1CSSc1cccc(CS(=O)(=O)Cc2ccccc2)c1]

### 3,5-dimethyl-4-({[3-(phenylmethanesulfonylmethyl)phenyl]disulfanyl}methyl)-1,2-oxazole

δH (400 MHz, CDCl3) 7.43-7.37 (7H, m), 7.31 (1H, dd, J=8.4, 7.6 Hz), 7.16 (1H, dm, J=7.7 Hz), 4.16 (2H, s), 4.06 (2H, s), 3.68 (2H, s), 2.23 (3H, s), 2.19 (3H, s)

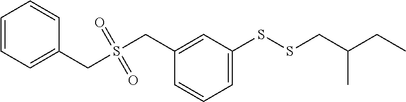 [SMILES: CCC(C)CSSc1cccc(CS(=O)(=O)Cc2ccccc2)c1]

### 1-[(2-methylbutyl)disulfanyl]-3-(phenylmethanesulfonylmethyl)benzene

δH (400 MHz, CDCl3) 7.53 (1H, dm, J=7.8 Hz), 7.49 (1H, t, 1.6 Hz), 7.43-7.36 (5H, m) overlapping 7.34 (1H, t, J=7.7 Hz), 7.22 (1H, dm, 7.7 Hz), 4.13 (2H, s), 4.08 (2H, s), 2.75 (1H, dd, J=12.8, 5.9 Hz), 2.59 (1H, dd, J=12.8, 7.5 Hz), 1.70 (1H, octet, J=6.6 Hz), 1.52-1.40 (1H, m), 1.26-1.11 (1H, m), 0.95 (3H, d, J=6.7 Hz), 0.83 (3H, t, J=7.4 Hz)

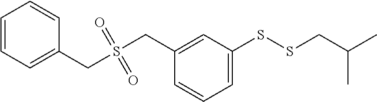 [SMILES: CC(C)CSSc1cccc(CS(=O)(=O)Cc2ccccc2)c1]

### 1-[(2-methylpropyl)disulfanyl]-3-(phenylmethanesulfonylmethyl)benzene

δH (400 MHz, CDCl3) 7.53 (1H, dm, J=7.9 Hz), 7.49(1H, t, J=1.6 Hz), 7.42-7.36 (5H, m) overlapping 7.34 (1H, t, J=7.7 Hz), 7.22 (1H, dm, J=7.6 Hz) overlapping residual CHCl3, 4.13 (2H, s), 4.08 (2H, s), 2.63 (2H, d, J=6.9 Hz), 1.92 (1H, nonet, J=6.7 Hz), 0.97 (6H, d, J=6.7 Hz)

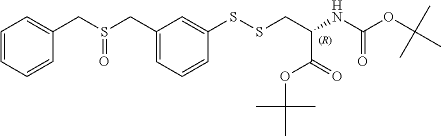

### tert-butyl(2R)-2-{[(tert-butoxy)carbonyl]amino}-3-{[3-(phenylmethanesulfinylmethyl)phenyl]disulfanyl}propanoate

δH (400 MHz, CDCl3) 7.49-7.42 (2H, m), 7.40-7.27 (6H, m), 7.15 (1H, dm, J=7.6 Hz), 5.38 (0.5H, br d, J=7.8 Hz), 5.33 (0.5H, br d, J=7.3 Hz), 4.48-4.38 (1 H, m), 3.94-3.78 (4H, m), 3.27, 3.26, 3.23, 3.22 (1H, ABX, J=14.1, 4.6 Hz), 3.18-3.08 (1H, m), 1.44 (9H, s), 1.41 (4.5H, br s) 1 1.40 (4.5H, br s)

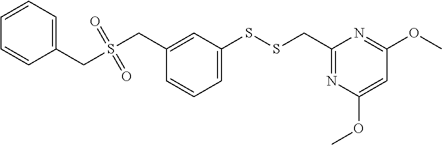 [SMILES: COc1cc(OC)nc(CSSc2cccc(CS(=O)(=O)Cc3ccccc3)c2)n1]

### 4,6-dimethoxy-2-({[3-(phenylmethanesulfonylmethyl)phenyl]disulfanyl}methyl)pyrimidine

δH (400 MHz, CDCl3) 7.46 (1H, dm, J=7.8 Hz), 7.42-7.34 (6H, m), 7.27 (1H, t, J=7.7 Hz), 7.19 (1H, double of broad triplets, J=7.6 Hz), 4.10 (2H, s), 4.03 (2H, s), 4.00 (2H, s), 3.83 (6H, s)

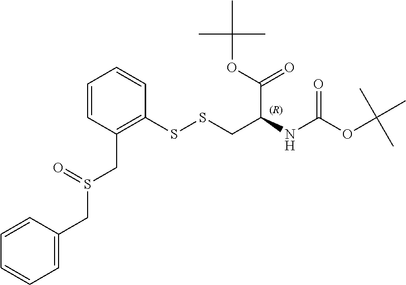

### tert-butyl(2R)-2-{[(tert-butoxy)carbonyl]amino}-3-{[2-(phenylmethanesulfinylmethyl)phenyl]disulfanyl}propanoate

δH (300 MHz, CDCl3): 1.45 (s, 9H, CH3)3); 1.47 (s, 9H, CH3)3), 3.00-3.28 (m, 2H, CH2-S—S), 3.90-4.10 (m, 2H, CH2-S—═O); 4.25-4.60 (m, 2H, CH2-S═O); 5.20-5.25 (m, 1H, —CH—NH); 7.25-7.84 (m, 9H, CH—Ar).

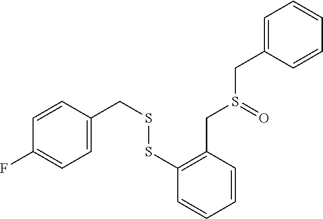 [SMILES: O=S(Cc1ccccc1)Cc1ccccc1SSCc1ccc(F)cc1]

### 1-fluoro-4-({[2-(phenylmethanesulfinylmethyl)phenyl]disulfanyl}methyl)benzene

δH (300 MHz, CDCl3): 3.82-4.04(m, 2H, CH2-S—S); 4.13-4.51 (m, 4H, CH2S═O), 7.05-7.70 (m, 13H, CH—Ar).

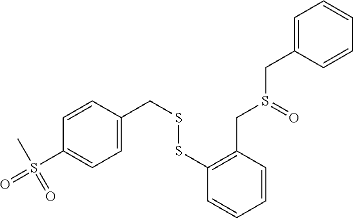 [SMILES: CS(=O)(=O)c1ccc(CSSc2ccccc2CS(=O)Cc2ccccc2)cc1]

### 1-methanesulfonyl-4-({[2-(phenylmethanesulfinylmethyl)phenyl]disulfanyl}methyl)benzene

δH (300 MHz, CDCl3): 3.09 (s, 3H, SO2CH3); 4.27 (m, 2H, CH2-S—S), 4.40 (dd, J.=12.6 Hz, 2H, CH2-S═O), 4.51 (dd, J.=12.9 Hz, 2H, CH2-S═O), 7.38-8.10 (m, 13H, CH—Ar).

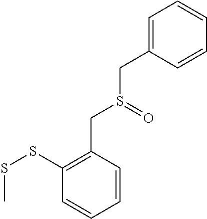 [SMILES: CSSc1ccccc1CS(=O)Cc1ccccc1]

### 1-(methyldisulfanyl)-2-(phenylmethanesulfinylmethyl)benzene

δH (300 MHz, CDCl3): 2.96 (s, 3H, CH3-S—S), 4.03 (dd, J.=12.6 Hz, 1H, CH2-S═O); 4.06 (d, J.=4.5 Hz, 2H, CH2-S═O); 4.44 (dd, J.=12.6 Hz, 1H, CH2-S═O), 7.28-7.75 (m, 9H, CH—Ar).

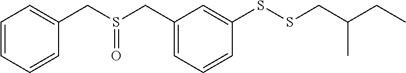 [SMILES: CCC(C)CSSc1cccc(CS(=O)Cc2ccccc2)c1]

### 1-[(2-methylbutyl)disulfanyl]-3-(phenylmethanesulfinylmethyl)benzene

δH (400 MHz, CDCl3) 7.46 (1H, dm, J=7.8 Hz), 7.42-7.34 (6H, m), 7.27 (1H, t, J=7.7 Hz), 7.19 (1H, double of broad triplets, J=7.6 Hz), 4.10 (2H, s), 4.03 (2H, s), 4.00 (2H, s), 3.83 (6H, s)

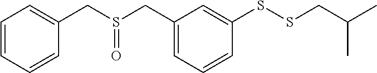 [SMILES: CC(C)CSSc1cccc(CS(=O)Cc2ccccc2)c1]

### 1-[(2-methylpropyl)disulfanyl]-3-(phenylmethanesulfinylmethyl)benzene

δH (400 MHz, CDCl3) 7.48 (1H, dm, J=7.9 Hz), 7.42-7.26 (7H, m), 7.12 (1H, doublet of broad triplets, J=7.6 Hz), 3.94, 3.91 (1H, AB, J=13.0 Hz), 3.90, 3.86 (1H, AB) overlapping 3.90, 3.86 (1H, AB), 3.82 (1H, AB, J=13.0 Hz), 2.61 (2H, d, J=6.9 Hz), 1.91 (1H, septet, J=6.7 Hz), 0.96 (6H, d, J=6.7 Hz)

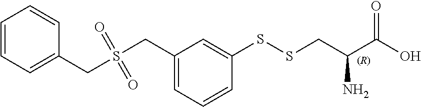

### (2R)-2-amino-3-{[3-(phenylmethanesulfonylmethyl)phenyl]disulfanyl}propanoic acid

δH (400 MHz, CD3OD) 7.70-7.67 (1H, m), 7.61 (1H, dm, J=7.8 Hz), 7.46-7.34 (7H, m), 4.44 (2H, s) overlapping 4.43 (2H, s), 4.30 (1H, dd, J=8.3, 4.3 Hz), 3.36 (1H, dd, J=14.9, 4.3 Hz), 3.19 (1H, dd, J=14.9, 8.2 Hz)

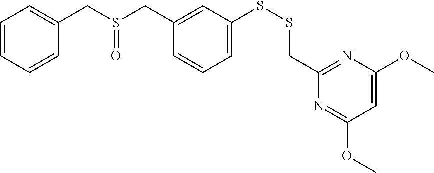 [SMILES: COc1cc(OC)nc(CSSc2cccc(CS(=O)Cc3ccccc3)c2)n1]

### 4,6-dimethoxy-2-({[3-(phenylmethanesulfinylmethyl)phenyl]disulfanyl}methyl)pyrimidine

δH (400 MHz, CDCl3) 7.42 (1H, dm, J=7.9 Hz), 7.39-7.25 (7H, m), 7.09 (1H, dm, J=7.6 Hz), 5.78 (1H, s), 3.99 (2H, s), 3.92, 3.89 (1H, AB, J=13.0 Hz), 3.87, 3.84 (1H, AB), overlapping 3.82 (6H, s), overlapping 3.83, 3.81 (1H, AB), 3.77, 3.74 (1H, AB, J=13.0 Hz)

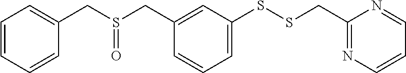 [SMILES: O=S(Cc1ccccc1)Cc1cccc(SSCc2ncccn2)c1]

### 2-({[3-(phenylmethanesulfinylmethyl)phenyl]disulfanyl}methyl)pyrimidine

δH (400 MHz, CDCl3) 8.56 (2H, d, J=4.9 Hz), 7.42-7.27 (7H, m), 7.21 (1H, t, J=7.7 Hz), 7.07 (1H, dm, J=7.6 Hz), 7.04 (1H, t, J=4.9 Hz), 4.20 (2H, s), 3.95, 3.92 (1H, AB, J=13.1 Hz), overlapping 3.91, 3.88 (1H, AB, J=13.1 Hz), 3.83 (1H, AB, J=12.9 Hz), 3.75 (1H, AB, J=13.0 Hz)

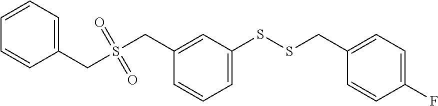 [SMILES: O=S(=O)(Cc1ccccc1)Cc1cccc(SSCc2ccc(F)cc2)c1]

### 1-fluoro-4-({[3-(phenylmethanesulfonylmethyl)phenyl]disulfanyl}methyl)benzene

δH (400 MHz, CDCl3) 7.44-7.36 (7H, m), 7.30-7.24 (1H, m), 7.23-7.14 (3H, m), 6.90 (2H, t, J=8.7 Hz), 4.14 (2H, s), 4.03 (2H, s), 3.90 (2H, s)

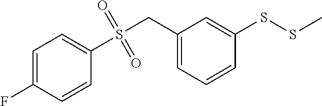 [SMILES: CSSc1cccc(CS(=O)(=O)c2ccc(F)cc2)c1]

### 1-(3-(((4-fluorophenyl)sulfonyl)methyl)phenyl)-2-methyldisulfane

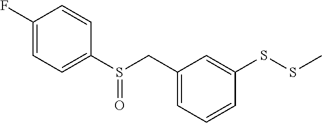 [SMILES: CSSc1cccc(CS(=O)c2ccc(F)cc2)c1]

### 1-(3-(((4-fluorophenyl)sulfinyl)methyl)phenyl)-2-methyldisulfane

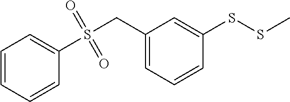 [SMILES: CSSc1cccc(CS(=O)(=O)c2ccccc2)c1]

### 1-methyl-2-(3-((phenylsulfonyl)methyl)phenyl)disulfane

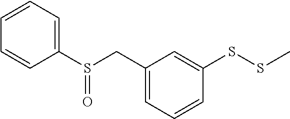 [SMILES: CSSc1cccc(CS(=O)c2ccccc2)c1]

### 1-methyl-2-(3-((phenylsulfinyl)methyl)phenyl)disulfane

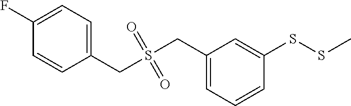 [SMILES: CSSc1cccc(CS(=O)(=O)Cc2ccc(F)cc2)c1]

### 1-(3-(((4-fluorobenzyl)sulfonyl)methyl)phenyl)-2-methyldisulfane

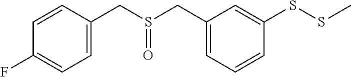 [SMILES: CSSc1cccc(CS(=O)Cc2ccc(F)cc2)c1]

### 1-(3-(((4-fluorobenzyl)sulfinyl)methyl)phenyl)-2-methyldisulfane

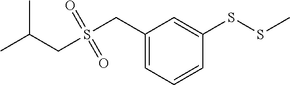 [SMILES: CSSc1cccc(CS(=O)(=O)CC(C)C)c1]

### 1-(3-((isobutylsulfonyl)methyl)phenyl)-2-methyldisulfane

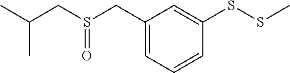 [SMILES: CSSc1cccc(CS(=O)CC(C)C)c1]

### 1-(3-((isobutylsulfinyl)methyl)phenyl)-2-methyldisulfane

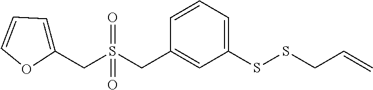 [SMILES: C=CCSSc1cccc(CS(=O)(=O)Cc2ccco2)c1]

### 2-({[3-(prop-2-en-1-yldisulfanyl)phenyl]methanesulfonyl}methyl)furan

There is further provided herein pharmaceutically acceptable salts, esters, and prodrugs of the compounds according to formula (I). The term “pharmaceutically acceptable salts, esters, and prodrugs” used herein refers to any pharmaceutically acceptable form of a compound of formula (I) (e.g. a salt or ester of a compound of formula (I)) which, upon administration to a patient, provides the compound of formula (I). Pharmaceutically acceptable salts include those derived from inorganic or organic bases and acids. Suitable salts include those derived from alkali metals such as potassium and sodium, and alkaline earth metals such as calcium and magnesium. Pharmaceutically acceptable prodrugs refer to a compound that is metabolized, for example hydrolyzed or oxidized, in the subject to form the compound according to formula (I).

There is further provided herein a pharmaceutical composition comprising a compound of formula (I) and a pharmaceutically acceptable carrier and/or excipient and/or diluent. The term “pharmaceutical composition” or “medicament” in the context of this invention means a composition comprising (a pharmaceutically effective amount of) a compound of formula (I) and additionally one or more pharmaceutically acceptable carriers and/or excipients and/or diluents.

The pharmaceutical composition may further contain ingredients selected from, for example, adjuvants, vehicles, preserving agents, fillers, binders, disintegrating agents, wetting agents, emulsifying agents, suspending agents, sweetening agents, flavouring agents, perfuming agents, lubricating agents, coating agents, encapsulating agents, aerosolising agents, and dispersing agents, depending on the nature of the mode of administration and dosage forms.

The pharmaceutical compositions may take the form, for example, of solid preparations including tablets, capsules, caplets, dragees, lozenges, granules, powders, pellets, beads, dressings, bandages, patches, surgical patches, catheters, pastes, and cachets; semi-solid preparations including gels, balms, creams, ointments, gums, foams, liniments, glues, and lotions; and liquid preparations including elixirs, syrups, suspensions, sprays, emulsions, lotions, soaps, shakes, collodions, paints, lavages, irrigates, and solutions; aerosol preparations using solutions, water based systems, suspensions, or dispersion systems, foam systems, and/or utilizing liquefied gas propellants, compressed gases, dry powder, and nebulisation. Techniques and formulations generally may be found in Remington, The Science and Practice of Pharmacy, Mack Publishing Co., Easton, Pa., latest edition.

The pharmaceutical composition (medicament) may, for example, be suitable for systemic, oral, inhalation, nasal, topical, suppository, intravenous or intradermal administration. The composition may alternatively be a nutraceutical composition, for example, a foodstuff, food supplement, dietary supplement, health supplement, meal replacement product, beverage, beverage supplement, food additive, animal feed or feed additive.

The compound of formula (I) may, for example, be administered in combination with one or more other biologically active agents. Thus, the pharmaceutical composition may comprise one or more other biologically active agents. The one or more biologically active agents may, for example, be selected from debridement agents; soaps; antibiotic agents such as penicillins (e.g. penicillin, amoxicillin), cephalosporins (e.g. cephalexin), macrolides (e.g. erythromycin, clarithromycin, azithromycin), flurooquinolones (e.g. ciproflaxcin, levoflacin, ofloxacin), sulphonamides (e.g. co-trimoxazole, trimethoprim), tetracyclines (e.g. tetracycline, doxycycline) and aminoglycosides (e.g. gentamicin, tobramycin); antiseptic agents such as taurolidine, potassium permanganate, boric acid, surfactants (e.g. octenidine dihydrochloride, octenidine dihydrochloride/phenoxyethanol), alcohols (e.g. ethanol, isoproyl alcohol, n-propanol), anilides (e.g. triclocarban), biguanides (e.g. chlorhexidine, polyhexadine, polyhexamethylene, polyhexanide), bisphenols (e.g. diphenyl ether—triclosan, chlorinated phenol—hexachlorophene), chlorine compounds (e.g. sodium hypochlorite), halophenols (e.g. chloroxylenol), iodine compounds (e.g. Lugol's solution, tincture of iodine, iodophores including polyvinylpyrrolidone iodine, povidone-iodine, cadexomer-iodine), silver compounds (e.g. silver sulphadiazine, silver nitrate, (irrespective of source i.e. silver released from solutions, creams ointments or nanocrystaline silver), peroxygens (e.g. hydrogen peroxide), and oxygen treatments (in the form of radical oxygen species and gaseous O₂ (e.g. hyperbaric chambers, Nitrox/Natrox); anti-inflammatory agents (e.g. nonsteroidal anti-inflammatory drugs (NSAIDs) such as aspirin, ibuprofen, and naproxen), anti-thrombotic agents such as anticoagulants (e.g. heparin or warfarin), antiplatelet drugs (e.g. aspirin), and thrombolytic drugs (e.g. streptokinase); antimicrobial light sources, wound healing devices, and photodynamic therapy sources within the ultraviolet, visible, violet, blue, green, yellow, red and infrared regions (singularly or a combination thereof), such as deep penetrating light therapy, low level light/laser therapy, utilizing light from such sources as lasers, wide or short range polarized and un-polarized light and incoherent light sources e.g. light emitting diodes (LED's).

The compound of formula (I) may, for example, be administered in combination with one or more other biologically active agents used for treating cystic fibrosis. Thus, the pharmaceutical composition may comprise one or more other biologically active agents used for treating cystic fibrosis. Examples of biologically active agents used for treating cystic fibrosis include, for example, modulators of cystic fibrosis transmembrane conductance regulators (CFTR) (e.g. ivacaftors, lumacaftor, tezacaftor), antibiotics, mucolytics (e.g. dornase alfa, hypertonic sodium chloride, mannitol), immunomodulatory drugs (e.g. azithromycin), bronchodilators, and steroid medicines.

In certain embodiments, the one or more other biologically active agent or agents are present in the composition or pharmaceutical composition in an amount ranging from about 0.00001 wt. % to about 99 wt. %, based on the total weight of the composition, for example, about 0.0001 wt.% to about 80 wt.% , or about 0.001 wt.% to about 50 wt. %, or about 0.1 wt. % to about 15 wt. %, or from about 0.5 wt. % to about 10 wt. %, or from about 0.5 wt. % to about 5 wt. %, or from about 0.1 wt. % to about 3 wt. %, or from about 0.1 wt. % to about 2 wt. %, or from about 0.1 wt. % to about 1 wt. %, or from about 0.001 wt. % to about 5 wt. %, or from about 0.001 wt. % to about 2 wt. %, or from about 0.001 wt. % to about 1 wt. %, or from about 0.001 wt. % to 20 about 0.5 wt. %, or from about 0.001 wt. % to about 0.1 wt. %, or from about 0.001 wt. % to about 0.01 wt. %.

In solid dosage forms for oral administration, the compound of formula (I) may be mixed with one or more pharmaceutically acceptable carriers, such as dicalcium phosphate or macrolides, and/or any of the following: diluents, fillers or extenders, such as, for example, starches, silicon lactose, sucrose, glucose, mannitol, microcrystalline cellulose and/or silicic acid; binders, such as, for example, hydroxypropylcellulose, hypromellose, hydroxypropyl methylcellulose, carboxymethylcellulose, gelatine, polyvinyl pyrrolidones, polyvinyl acetate, sucrose and/or acacia; disintegrating agents, such as starch, for example, potato or tapioca starch, starch derivatives such as sodium starch glycolate, crospolyvinylpyrollidone, calcium carbonate, croscarmellose soduium, alginic acid, and certain silicates; lubricants, such as talc, calcium stearate, magnesium stearate, stearic acid, sodium sulfate stearyl fumarate, solid polyethylene glycols, solubilisers such as sodium lauryl sulfate, ammonium dodecyl sulfate and sodium dodecyl sulfate; surfactants such as non-ionic surfactants, for example, polyglycerol alkyl ethers, glucosyl dialkyl ethers, crownethers; ester-linked surfactants, for example, polyoxyethylene alkyl ethers, Brij, Spans (sorbitan esters) and Tweens (Polysorbates); anionic surfactants, for example, sulfonate, phosphate, sulfate and carboxylates; alkyl carboxylates (soaps), for example, sodium stearate, dioctyl sodium sulfosuccinate, perfluorooctanesulfonate, linear alkylbenzene sulfonates (LABs) and perfluorobutanesulfonate; alkyl-aryl ether phosphates, sodium lauroyl sarcosinate and carboxylate-based fluorosurfactants, for example, perfluorononanoate and perfluorooctanoate; cationic surfactants, for example, benzalkonium chloride, cetylpyridinium chloride, and benzethonium chloride; alkyltrimethylammonium salts, for example, cetyl trimethylammonium bromide (CTAB) and cetyl trimethylammonium chloride (CTAC); zwitterionic surfactants, for example, sulfonates, as in the sultaines CHAPS (3-[(3-Cholamidopropyl)dimethylammonio]-1-propanesulfonate); betaines, for example, cocamidopropyl betadine; flavouring and colouring agents and mixtures thereof. Tablets, and other solid dosage forms of the pharmaceutical compositions, may optionally be prepared with coatings and shells, such as enteric coatings and other coatings well known in the pharmaceutical-formulation art. They may also be formulated so as to provide slow or controlled release of the active ingredient(s) therein using, for example, natural and synthetic polymers such as hydroxypropylmethyl cellulose methacrylates, methacrylic acid copolymers (e.g. methyl acrylate-methacrylic acid copolymers and methyl methacrylate-methacrylic acid copolymers), shellac, ethylcellulose, cellulose acetate phthalate, cellulose acetate trimellitate, polyvinyl acetate phthalate, cellulose acetate succinate, hydroxyl propyl methyl cellulose acetate succinate, sodium alginate, waxes, fatty acids, zein, respectively, in varying proportions to provide the desired release profile, other polymer matrices, liposomes and/or microspheres may also be used. These compositions may also optionally contain colourants and/or opacifying agents and may be of a composition such that they release the active ingredient(s) only, or preferentially, in a certain portion of the gastrointestinal tract, optionally, in a delayed manner.

The pharmaceutical compositions may comprise no more than about 50% w/w of pharmaceutically acceptable carrier and/or excipient and/or diluent, for example, no more than about 45% w/w of pharmaceutically acceptable carrier and/or excipients and/or diluents, or no more than about 40% of w/w pharmaceutically acceptable carrier and/or excipients and/or diluents, or no more than about 35% w/w of pharmaceutically acceptable carrier and/or excipients and/or diluents. For example, the pharmaceutical composition may comprise at least about 1% w/w, or at least about 10% w/w, or at least about 15% w/w, or at least about 20% w/w, or at least about 25% w/w, or at least about 30% w/w of pharmaceutically acceptable carrier and/or excipients and/or diluents.

Liquid form preparations include solutions, suspensions, and emulsions, for example, water or water-propylene glycol solutions for oral administration. Liquid preparations can also be formulated in solution in aqueous polyethylene glycol solution. In certain embodiments, the compound of formula (I) may be mixed with one or more pharmaceutically acceptable carriers, such as water and/or any of the following: solvent such as propylene glycol, alcohol; humectant such as glycerol; sweeteners such as liquid glucose, corn syrup and sucrose; artificial sweeteners such as aspartame, stevia and sucralose; preservatives such as benzoates and parabens; viscosity modifiers/thickeners such as gums and alginates; buffering agents; flavouring agents and colouring agents.

Also included are solid form preparations, for example, tablets, capsules, granules and powder, which are intended to be converted, shortly before use, to liquid form preparations for oral administration. Such liquid forms include solutions, suspensions, and emulsions. These particular solid form preparations are most conveniently provided in unit dose form and as such are used to provide a single liquid dosage unit. Alternatively, sufficient solid may be provided so that multiple individual liquid doses may be reconstituted when required, by measuring predetermined volumes of the solid form preparation as with a spoon, or other measuring device. The solid form preparations intended to be converted to liquid form may contain, in addition to the active material, flavourings, colourants, stabilizers, buffers, artificial and natural sweeteners, dispersants, thickeners, solubilising agents, and the like. The liquid utilized for preparing the liquid form preparation may be water, isotonic water, juices, milk, ethanol, and the like as well as mixtures thereof.

The components referred to hereinbefore and hereinafter are in particular selected from among those such as are listed in pharmacopoeia, e.g. in the US Pharmacopoeia National Formulary, the Pharmacopoea Europea, the Pharmacopoea Helvetica, the British Pharmacopoeia, the German Pharmacopoeia, the Chinese Pharmacopoeia, the Japanese Pharmacopoeia, or supplements, such as by way of decrees.

Uses of the Compounds of Formula (I) and Pharmaceutical Compositions

The compounds and pharmaceutical compositions described herein may be used in various therapeutic and non-therapeutic applications. For example, the compounds and pharmaceutical compositions described herein may be used to provide one or more beneficial effects to a patient. For example, the compounds and pharmaceutical compositions described herein may be used in various cosmetic applications. For example, the compounds and pharmaceutical compositions described herein may be used in an in vitro method or in an in vivo method. The methods may comprise administering the compound or pharmaceutical composition described herein to a subject.

The term “therapeutic treatment” or “therapeutic method”, also includes prophylaxis and the alleviation of symptoms of a disease and/or disorder in a subject, although not cosmetic treatments.

The expression “treating or preventing” and analogous terms used herein refers to all forms of healthcare intended to remove or avoid the disease and/or disorder or to relieve its symptoms, including preventive and curative care, as judged according to any of the tests available according to the prevailing medical practice. An intervention that aims with reasonable expectation to achieve a particular result but does not always do so is included within the expression “treating or preventing”. An intervention that succeeds in slowing or halting progression of a disease and/or disorder is included within the expression “treating or preventing”.

In certain embodiments, the subject is a human. In other embodiments, the subject is a mammal other than a human, such as non-human primates (e.g. apes, monkeys and lemurs), companion animals such as cats or dogs, working and sporting animals such as dogs, horses and ponies, farm animals such as pigs, sheep, goats, deer, oxen and cattle, and laboratory animals such as rodents (e.g. rabbits, rats, mice, hamsters, gerbils or guinea pigs).

The amount of compounds or pharmaceutical composition administered may be varied depending upon the requirements of the subject or use. For both therapeutic and non-therapeutic applications, the amount of compound or pharmaceutical composition administered may be varied depending upon the desired results, the requirements of the subject and the severity of the condition being treated. Determination of the proper amount/dosage for a particular situation is within the skill of the art. For example, for therapeutic applications a physician or veterinarian having ordinary skill in the art can readily determine and prescribe the effective amount of the compound or pharmaceutical composition required. The total daily amount/dosage may be divided and administered in portions during the day if desired.

In general, a suitable daily dose of active agents (i.e. compounds of formula (I)) will be that amount which is the lowest dose effective to produce the desired effect, for example, a therapeutic effect. It is contemplated that a wide range of doses may be used, due to the non-toxic nature of the composition. A person of ordinary skill in the art will understand that a suitable dose or dosage will typically vary from subject to subject, and will dependent on factors such as the severity of health conditions of the subject at the outset of administration. For example, the dose of active agents in the composition may be up to 15 g per day, for example, up to about 10 g per day, or up to about 5 g per day. In certain embodiments, the doses of active agents is in the range of 100 mg to about 3 g per day, which may be administered as two or three or more sub-doses administered separately at appropriate intervals throughout the day, optionally in unit dosage forms. In certain embodiments, the dose of active agents in the composition may be from about 200 mg to about 3 g of the compound per day, for example, from about 500 mg to about 3 g of the compound per day, or from about 750 mg to about 2.5 g of the compound per day, or from about 1000 mg to about 2000 mg of the compound per day. In certain embodiments, the active agent may be administered two or three times a day. In certain embodiments, each dose of active agents is no more than about 5 g, for example, no more than about 3 g, for example, no more than about 2.5 g. Each dose of the active agents may be combined with other conventional agents for the desired effect. Where the composition is for topical administration, the concentration of the compound may be from about 0.01 g to about 0.5 g per cm² of skin, or from about 0.1 g to about 0.4 g or from about 0.2 g to about 0.3 g per cm² of skin.

The compounds and pharmaceutical compositions described herein may, for example, be used for treating a microbial infection and/or for treating inflammation and/or for reducing the formation of blood clots and/or for treating a wound and/or for treating cystic fibrosis. These uses may, for example, be therapeutic or non-therapeutic. For example, the compounds and pharmaceutical compositions described herein may be used treat or prevent a chronic wound in a subject. For example, the compounds and pharmaceutical compositions described herein may be used treat or prevent an ischaemic ulcer a subject. For example, the compounds and pharmaceutical compositions described herein may be used to reduce mucus build-up in the lungs of a subject having cystic fibrosis.

The compounds and pharmaceutical compositions described herein may be used as an antimicrobial. As used herein, the term “antimicrobial” means that the compositions and pharmaceutical compositions described herein may be used to kill microbes, to inhibit bacterial motility, for example swarming, swimming or twitching, and/or to inhibit the growth of microbes and/or to reduce the growth of microbes. The term “antimicrobial” includes the effect of decreasing virulence of microbes such as bacteria and inhibiting quorum-sensing.

The compounds and pharmaceutical compositions described herein may be used to inhibit and/or eradicate bacterial biofilm formation and/or to disrupt a bacterial biofilm that has already formed. The inhibition and/or eradication of bacterial biofilm formation and/or the disruption of a bacterial biofilm may, for example, be the result of the inhibition of bacterial signalling, for example quorum sensing, inhibition of the formation or dispersal of bacterial biofilms, inhibition or stimulation of global regulatory systems (for example those dependent upon intracellular levels of cyclic dimeric guanosine monophosphate), and inhibition of the formation of functional bacterial cell surface and excreted proteins.

In certain embodiments, the microbes may be selected from bacteria, fungi, and protozoa. The bacterial strains may, for example, be selected from gram positive bacteria, gram negative bacteria, atypical bacteria, and diderm bacteria. The gram positive bacteria may, for example, be selected from one or more of Clostridium perfringens, Listeria monocytogenes, Bacillus cereus, Enterococcus faecalis, Staphylococcus aureus, Methicillin-resistant Staphylococcus aureus, Staphylococcus epidermidis, Streptococcus pneumoniae, Streptococcus pyogenes, Clostridium spp., and Peptostreptococcus spp. The gram negative bacteria may, for example, be selected from one or more of Salmonella Typhimurium Vibrio parahaemolyticus, Enterobacteriae, for example, Escherichia coli, Klebsiella pneumoniae, Enterobacter spp., Proteus spp., Citrobacter spp., Morganella morgannii, Pseudomonas aeruginosa, Acinetobacter baumannii, Campylocateri jejuni, Bacteriodes spp., Prevotella spp., Helicobacter pylori, and Porphyromonas spp. The atypical bacteria may, for example, be selected from one or more of Mycoplasma pneumonia, Chlamydia pneumonia, Legionella pneumophila, and mycobacteria. The diderm bacteria may, for example, be a Spirochaete bacteria, for example selected from Leptospira species, Borrelia burgdorferi, Borrelia mayonii, Borrelia afzelii, Borrelia garinii, Borrelia recurrentis, Treponema pallidum, Brachyspira pilosicoli, and Brachyspira aalborgi. The fungal strains may, for example, be selected from one or more of Candida albicans, Candida glabrata, Aspergillus fumigatus.

The bacterial strains may be a biofilm forming bacteria and/or a bacteria capable of forming biofilm. The biofilm forming bacteria and/or bacteria capable of forming biofilm may, for example, be selected from Bacillus spp, Listeria monocytogenes, Staphylococcus aureus, Lactobacillus plantarum, Lactococcus lactis, Vibrio fischeri, Aeromonas hydrophila, Aeromonas salmonicida, Agrobacterium tumefaciens, Burkholderia cepacia, Chromobacterium violaceum, Enterobacter agglomerans, Erwinia carotovora, Erwinia chtysanthemi, Erwinia Stewartii, Escherichia coli, Helicobacter pylori, Pseudomonas aureofaciens, Pseudomonas aeruginosa, Ralstonia solanacearum, Rhizobium etli, Rhizobium leguminosarum, Rhodobacter sphaeroides, Salmonella typhimurium, Serratia liquefaciens, Sinorhizobium meliloti, Vibrio anguillarum, Vibrio harveyi, Yersinia enterocolitica, Yersinia pseudotuberculosis.

For example, the compounds and pharmaceutical compositions described herein may be administered to a subject to treat and/or prevent a microbial infection in a subject. For example, the compounds and pharmaceutical compositions described herein may be used to facilitate healing of damaged wound on the skin. For example, the compounds and pharmaceutical compositions described herein may be used to prevent microbial infection of damaged skin. For example, the compounds and pharmaceutical compositions described herein may be used to treat or prevent a microbial infection in the digestive system. For example, the compounds and pharmaceutical compositions described herein may be used to treat or prevent a microbial infection in the nasal or aural cavity of a subject. For example, the compounds and pharmaceutical compositions described herein may be used treat or prevent a microbial infection in the respiratory tract of a subject. For example, the compounds and pharmaceutical compositions described herein may be used treat or prevent a microbial infection in the respiratory tract (e.g. lungs) of a cystic fibrosis patient (e.g. by inhalation of a compound of formula (I)). For example, the compounds and pharmaceutical compositions described herein may be used treat or prevent a microbial infection in the skin of a subject. For example, the compounds and pharmaceutical compositions described herein may be used treat or prevent a chronic wound in a subject. For example, the compounds and pharmaceutical compositions described herein may be used treat or prevent an ischaemic ulcer a subject. For example, the compounds and pharmaceutical compositions described herein may be used as a urinary tract rinse or a bladder rinse, for example for urinary tract implant, indwelling urinary catheter, and kidney dialysis patients.

Thus, there is provided herein a therapeutic use of a compound or pharmaceutical composition described herein as an antimicrobial. There is also provided herein a compound or pharmaceutical composition as described herein for use as an antimicrobial. There is further provided herein a use of a composition or pharmaceutical composition as described herein in the manufacture of an antimicrobial medicament. There is further provided herein a therapeutic method for treating and/or preventing a microbial infection in a subject, the method comprising administering a compound or pharmaceutical composition as described herein to the subject.

In certain embodiments, the compounds and pharmaceutical compositions disclosed herein are used in non-therapeutic applications.

For example, the compounds described herein may be used as an antimicrobial agent on non-living surfaces (e.g. as a disinfectant). For example, the compounds and pharmaceutical compositions disclosed herein may be used for cosmetic applications, for example as an antimicrobial agent on living surfaces (e.g. skin). For example, the compounds and pharmaceutical compositions disclosed herein may be used as an antimicrobial in cosmetic skincare compositions or makeup compositions.

For example, the compounds described herein may be used as an antimicrobial agent on industrial non-living surface, for example to remove or prevent biofilm formation in piping used in the water or oil industries, as a cleaning agent for water tanks (e.g. in fisheries), as an anti-fowling agent in industrial processes (e.g. water industries, fisheries).

For example, the compounds described herein may be used as food and/or water additives for preservation and/or prevention of disease transmission. For example, the compounds described herein may be used in plant, fresh fruit and vegetable washes. The compounds described herein may reduce surface bacteria, extend shelf life and/or protect the surface from pest invasion in live crops or agricultural produce.

For example, the compounds described herein may be used as an antimicrobial in medical devices or medical compositions, for example in cements for bone or dental implants, in implants, in wound dressings, in stitches, or in threads.

For example, the compounds described herein may be used as an antimicrobial on living surface. For example, the compounds described herein may be applied to the skin to kill microbes or inhibit growth of microbes for hygiene reasons (e.g. to prevent spread of disease). For example, the compounds described herein may be applied to the hands as a hand sanitizer. For example, the compounds described herein may be used as an oral rinse, for example to treat or prevent halitosis. For example, the compounds described herein may be used as a dental care product, for example to treat or prevent cavities and plague.

For example, the compounds described herein may be used for agricultural applications. For example, the compounds described herein may be used to treat or prevent infection of plant micro-wounds or may be used to reduce surface pathogens on a plant. For example, the compounds described herein may be used as bio-security sanitizer, for example for animal farm facilities. For example, the compounds described herein may be used for animal feed sterilization.

The compounds and pharmaceutical compositions described herein may be used as an anti-inflammatory agent. A used herein, the term “anti-inflammatory” means that the compositions and pharmaceutical compositions described herein may be used to reduce inflammation or swelling.

For example, the compounds and pharmaceutical compositions described herein may be administered to a subject to treat and/or prevent inflammation in a subject. Thus, there is provided herein a therapeutic use of a compound or pharmaceutical composition described herein as an anti-inflammatory. There is also provided herein a compound or pharmaceutical composition as described herein for use as an anti-inflammatory. There is further provided herein a use of a composition or pharmaceutical composition as described herein in the manufacture of an anti-inflammatory medicament. There is further provided herein a therapeutic method for treating and/or preventing inflammation in a subject, the method comprising administering a compound or pharmaceutical composition as described herein to the subject.

The compounds and pharmaceutical compositions described herein may therefore be used to treat or prevent an inflammatory disease or disorder. Examples of inflammatory diseases or disorders include, for example, allergies, cancer, atherosclerosis, ischemic heart disease, acne vulgaris, asthma, autoimmune diseases, autoinflammatory diseases, celiac disease, chronic prostatitis, colitis, diverticulitis, glomerulonephritis, hidradenitis suppurativa, hypersensitivities, inflammatory bowel diseases, interstitial cystitis, lichen planus, mast cell activation syndrome, mastocytosis, otitis, pelvic inflammatory disease, reperfusion injury, rheumatic fever, rheumatoid arthritis, rhinitis, sarcoidosis, transplant rejection, and vasculitis.

Treatment of inflammation may, for example, be determined by measuring the expression level of one or more inflammation markers such as, for example, C-reactive protein (CRP) and erythrocyte sedimentation rate (ESR). A reduction in CRP and ESR expression indicates a reduction of inflammation.

The compounds and pharmaceutical compositions described herein may be used as an anti-thrombotic agent. A used herein, the term “anti-thrombotic” means that the compositions and pharmaceutical compositions described herein may be used to reduce the formation of blood clots, for example by inhibiting platelet aggregation. The anti-thrombotic may therefore be used to increase blood flow in a subject.

For example, the compounds and pharmaceutical compositions described herein may be administered to a subject to reduce the formation of blood clots in a subject. Thus, there is provided herein a therapeutic use of a compound or pharmaceutical composition described herein as an anti-thrombotic. There is also provided herein a compound or pharmaceutical composition as described herein for use as an anti-thrombotic. There is further provided herein a use of a composition or pharmaceutical composition as described herein in the manufacture of an anti-thrombotic medicament. There is further provided herein a therapeutic method for reducing the formation of blood clots in a subject, the method comprising administering a compound or pharmaceutical composition as described herein to the subject.

The compounds and pharmaceutical compositions described herein may therefore be used to treat or prevent a disorder associated with blood flow in a subject. The compounds and pharmaceutical compositions disclosed herein may be used to improve (e.g. increase) blood flow in a subject, for example in a subject that has a blood flow level that is outside what is considered to be a normal or healthy range. For example, the compounds and pharmaceutical compositions disclosed herein may be used to treat or prevent any disease or disorder associated with blood flow (e.g. circulation system) in a subject. For example, the subject may be susceptible to developing one or more diseases or disorders associated with blood flow.

Diseases and disorders associated with blood flow and/or platelet aggregation include, for example, cardiovascular diseases, cerebrovascular or brain disease, immune diseases, bone, joint and/or muscle diseases and fatigue diseases.

Cardiovascular diseases and disorders include, for example, coronary artery diseases, coronary heart disease, angina, myocardial infarction, hypertensive heart disease, heart failure, pulmonary heart disease, cardiac dysrhythmias, inflammatory heart disease, rheumatic heart disease, cardiomyopathy, atrial myopathy, congenital heart disease, endocarditis, inflammatory cardiomegaly, myocarditis, valvular heart disease, aortic aneurysms, venous thrombosis, rheumatoid vasculitis, atherosclerosis peripheral artery diseases and renal artery stenosis.

Cerebrovascular diseases and brain diseases include, for example, stroke (e.g. mini-stroke, hemorrhagic stroke, ischemic stroke), transient ischemic attack (TIA), subarachnoid haemorrhage and vascular dementia.

Immune diseases and disorders include, for example, any disease in which the subject's immune response is lower than a normal healthy individual, for example immunodeficiency diseases (e.g. primary, secondary, humoral, T cell, neutropenia, asplenia and complement deficiency diseases), low immune response in subjects whose immune system has been compromised (e.g. subjects undergoing chemotherapy and/or radiotherapy, subjects infected with HIV, subjects missing one or more organs associated with immune function such as the spleen, tonsils, lymph nodes). For example, immune diseases include ataxia-telangiectasia, Chediak-Higashi syndrome, combined immunodeficiency disease, complement deficiencies, DiGeorge syndrome, hypogammaglobulinemia, Job syndrome, leukocyte adhesion defects, panhypogammaglobulinemia, Bruton's disease, congenital agammaglobulinemia, selective deficiency of IgA, Wiskott-Aldrich syndrome and severe combined immunodeficiency disorder (SCID). For example, infection or symptoms related to immune health include upper and lower respiratory tract infection, hay fever, sinus and pharyngitis.

Joint diseases and disorders include, for example, any disease related to joints, mobility, muscle and bone health, for example arthritis (e.g. osteoarthritis, rheumatoid arthritis, psoriatic arthritis, septic arthritis), bursitis, osteonecrosis, dislocations, Perthes disease and Paget's disease of the bone.

Fatigue diseases and disorders include, for example, simple fatigue and/or any disease in which fatigue is a symptom. For example, fatigue diseases and disorders include chronic fatigue syndrome, anaemia, depression, iron deficiency (without anaemia), sleep disorders, underactive thyroid, overactive thyroid, Addison disease, anorexia nervosa or other eating disorders, autoimmune diseases such as lupus, diabetes, fibromyalgia, kidney disease, liver disease and malnutrition.

In certain embodiments, the compound or pharmaceutical composition described herein may be used for maintaining and/or improving the overall health of the circulatory system, for example to treat or reduce or prevent the onset of one or more circulatory system-associated diseases or disorders, and/or to provide beneficial effects to the metabolic system via the maintenance or improvement of healthy blood flow.

The compounds and pharmaceutical compositions described herein may be used in various non-therapeutic applications.

For example, the compounds and pharmaceutical compositions may be used in methods of maintaining blood flow in a subject. For example, where a subject is healthy or has a normal blood flow level, and may, for example, not be at any particular risk of developing any disease associated with blood flow, the subject may consume the compositions disclosed herein to maintain a normal blood flow level. For example, where a subject is healthy and may, for example, not be at any particular risk of developing any disease associated with blood flow and/or platelet aggregation, the subject may consume the compositions disclosed herein as part of a healthy lifestyle.

The anti-thrombotic activity of a compound may, for example, be measured by determining the degree of platelet aggregation, for example using an optical aggregometer.

The compounds and pharmaceutical compositions described herein may be used as an anti-cancer agent. Thus, the compounds and pharmaceutical compositions described herein may therefore be used to treat or prevent cancer.

Methods for Making a Compound of Formula (I)

The compounds of formula (I) and the pharmaceutical compositions comprising a compound of formula (I) may be made by any suitable method.

In certain embodiments, the pharmaceutical compositions are made by combining a compound of formula (I) with a pharmaceutically acceptable carrier and/or excipient and/or diluent. The components are combined in suitable amounts to obtain a composition having the desired quantity and concentration of each component. Each component may be combined with one or more other components in any order and combination suitable to obtain the desired product. For example, each component may be combined by mixing. Such methods are well known in the art, for example, methods known in the pharmaceutical industry. The pharmaceutical composition may be prepared in the dry solid form, for example, powder form, and subject to further processing steps depending on the types of the formulation for the intended finished products. The methods may further comprise a forming step, wherein the mixture is moulded, pressed, spray dried or otherwise formed into a shape (e.g. bar, ball, pellet, clusters, tablet), preferably with dimensions and/or textures suitable for consumption by a human or other mammalian animal of the types described herein.

In certain embodiments, the method for making the compounds of formula (I) may proceed via steps 1 to 4 or steps 1 to 5 of reaction scheme (IA) or reaction scheme (IB). Reaction scheme (IA) may, for example, make compounds according to formula (IA). Reaction scheme (IB) may, for example, make compounds according to formula (IB).

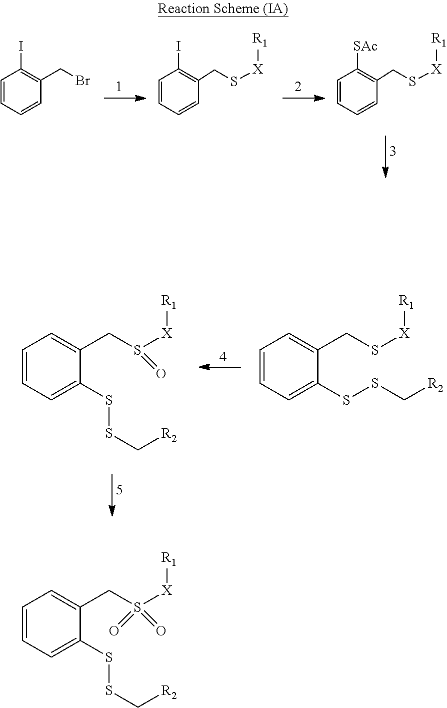

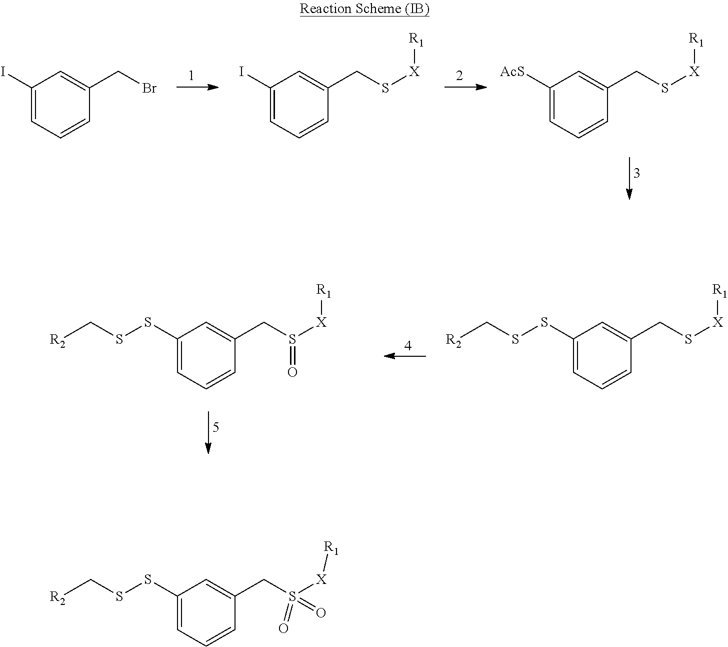

Step 1 may comprise reacting

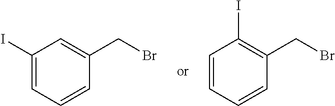 [SMILES: BrCc1cccc(I)c1 ; BrCc1ccccc1I]

with R¹-mercaptan. For example, step 1 may comprise reacting

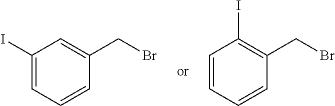 [SMILES: BrCc1cccc(I)c1 ; BrCc1ccccc1I]

with alkyl mercaptan or substituted thiol, for example benzyl mercaptan.

The reaction of step 1 may, for example, take place in the presence of a hydride such as sodium hydride. The reaction of step 1 may, for example, take place in the presence of a heterocyclic compound such as a cyclic ether such as tetrahydrofuran.

The reaction of step 1 may, for example, take place at a temperature ranging from about 15° C. to about 25° C. The reaction of step 1 may, for example, take place for a period of time ranging from about 6 hours to about 24 hours.

Purification of the product of step 1 may, for example, comprise quenching the reaction mixture, for example with ammonium chloride, and removing any volatiles. The residue may then be partitioned between water and an organic compound such as toluene. The aqueous layer may, for example, be extracted for a second time using more organic compound such as toluene. The organic layers may then be combined and/or dried, for example over sodium sulphate. The product may then be filtered and/or concentrated, for example under reduced pressure. The crude residue may be purified by silica gel flash column chromatography, for example using a 80 g or 120 g silica cartridge and heptane:dichloromethane 0-10% as the solvent system.

Step 2 may comprise reacting

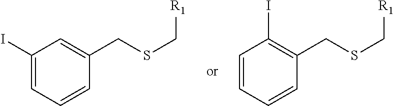

with a thioacetate such as potassium thioacetate, for example in the presence of an organic compound such as toluene. Nitrogen may, for example, be bubbled through the mixture. A metal halide such as copper iodide and/or a heterocyclic compound such as 1,10 phenanthroline may be added to the reaction mixture. The reaction may, for example, take place under reflux conditions. The reaction may, for example, take place at a temperature ranging from about 80° C. to about 120° C., for example about 100° C. The reaction may, for example take place for a period of time ranging from about 12 hours to about 36 hours, for example about 48 hours.

Purification of the product of step 2 may, for example, comprise cooling the reaction mixture to approximately room temperature. The reaction may, for example, be partitioned between water and an organic compound such as ethyl acetate. The aqueous layer may, for example, be extracted for a second time using more organic compound such as ethyl acetate. The organic layers may then be combined and/or dried, for example over sodium sulphate. The product may then be filtered and/or concentrated, for example under reduced pressure. The crude residue may be purified by silica gel flash column chromatography, for example using a 80 g silica cartridge and heptane:dichloromethane 10-20% as the solvent system.

Step 3 may comprise reacting

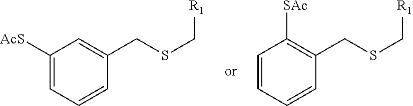

with a sulfonothioate including a —CH₂—R₂ group, for example S-benzyl 4-methylbenzenesulfonothioate. The reaction may, for example, take place in the presence of a hydroxide such as potassium hydroxide and/or one or more organic compounds such as an alcohol such as methanol and/or a heterocyclic compound such as a cyclic ether such as tetrahydrofuran. The reaction may, for example, take place at a temperature equal to or less than about 0° C., for example equal to or less than about −20° C. or equal to or less than about −40° C. or equal to or less than about −60° C. The reaction mixture may, for example, be cooled using an acetonitrile/cardice bath or an acetone/cardice bath. The reaction may, for example, take place for a period of time ranging from about 15 minutes to about 2 hours, for example about 1 hour.

Purification of the product of step 3 may, for example, comprise neutralising the reaction mixture, for example with an aqueous solution of a salt such as ammonium chloride. The reaction mixture may be partitioned between an organic compound such as ethyl acetate and an aqueous solution of a salt such as ammonium chloride. The aqueous layer may, for example, be extracted for a second time using more organic compound such as ethyl acetate. The organic layers may then be combined and/or dried, for example over sodium sulphate. The product may then be filtered and/or concentrated, for example under reduced pressure. The crude residue may be purified by silica gel flash column chromatography, for example using a 120 g silica cartridge and heptane:ethyl acetate 1-2% as the solvent system.

Step 4 may comprise reacting

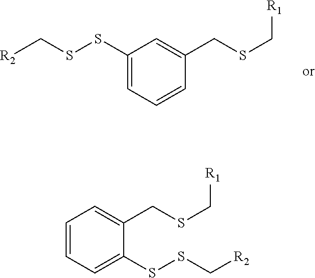

with an oxidising agent such as 3-chloroperbenzoic acid. The reaction may, for example, take place in the presence of an organic solvent such as dichloromethane.

The reaction may, for example, take place at a temperature equal to or less than about 0° C., for example equal to or less than about −20° C. or equal to or less than about −40° C. or equal to or less than about −60° C. The reaction may, for example, take place for a period of time ranging from about 15 minutes to about 2 hours, for example about 1 hour.

Purification of the product of step 4 may, for example, comprise washing the reaction mixture, for example with an aqueous solution of sodium bicarbonate. The aqueous layer may, for example, be further extracted with an organic solvent such as dichloromethane. The organic layers may then be combined and/or dried, for example over sodium sulphate. The product may then be filtered and/or concentrated, for example under reduced pressure. The crude residue may be purified by silica gel flash column chromatography, for example using a 20 g silica cartridge and heptane:ethyl acetate 30-40% as the solvent system.

Step 5 may comprise reacting

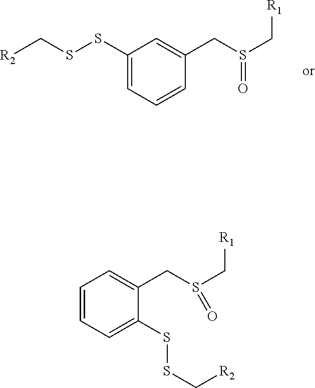

with an oxidising agent such as potassium permanganate. The reaction may take place in the presence of an organic solvent such as acetone. The reaction may, for example, take place at a temperature equal to or less than about 0° C., for example equal to or less than about −20° C. or equal to or less than about −40° C. The reaction may, for example, take place for a period of time ranging from about 15 minutes to about 10 hours, for example about 6 hours.

Purification of the product of step 5 may, for example, comprise filtering through a pad of celite. The solvent may, for example, be removed and the crude material may, for example, be purified via silica gel flash column chromatography, for example using a 12 g silica cartridge and heptane:ethyl acetate 10-15% or hexane:dichloromethane as the solvent system.

### EXAMPLES

### Example 1

### Method of preparation of 1-Benzyl-2-(3-((benzylsulfinyl)methyl)phenyl)disulfane and 1-benzyl-2-(3-(benzylsulfonyl)phenyl)disulfane

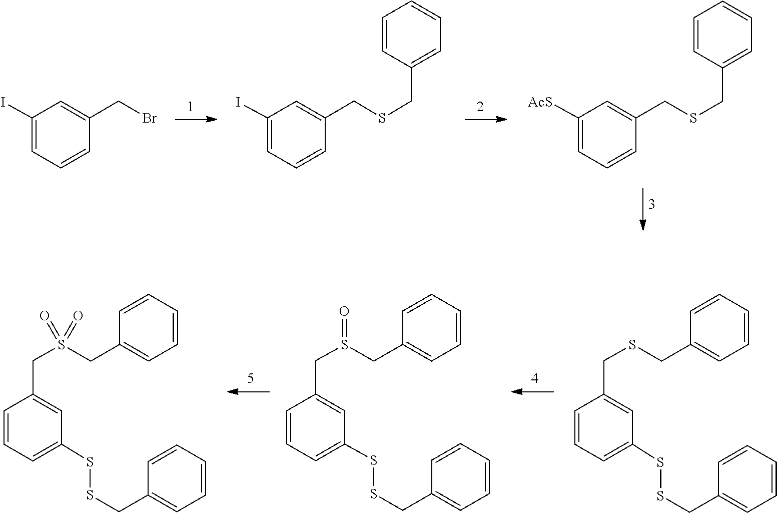

### Step 1—Preparation of Benzyl(3-iodobenzyl)sulfane

Benzyl mercaptan (2.17 mL, 18.50 mmol) was added dropwise to a suspension of sodium hydride (60% in mineral oil) (808 mg, 20.20 mmol) in tetrahydrofuran (100 mL), over a period of 5 minutes, followed by the addition of a solution of 3-bromomethyl iodobenzene (5.00 g, 16.80 mmol) in tetrahydrofuran (15 mL). The resulting mixture was stirred at room temperature overnight.

The reaction mixture was quenched with a saturated aqueous solution of ammonium chloride and volatiles were removed in vacuo. The residue was partitioned between water (100 mL) and toluene (100 mL). The aqueous layer was further extracted with toluene (2×250 mL). The organic layers were combined, dried over sodium sulfate, filtered and concentrated under reduced pressure. The crude residue was purified via silica gel flash column chromatography using an 80 g silica cartridge and heptane:dichloromethane 0-10% as solvent system in order to afford the desired compound, benzyl(3-iodobenzyl)sulfane, as a red oil (5.49 g, 96% yield).

### Step 2—Preparation of S-(3-((Benzylthio)methyl)phenyl) ethanethioate

Potassium thioacetate (1.24 g, 10.90 mmol) was added to a solution of benzyl(3-iodobenzyl)sulfane (3.70 g, 10.90 mmol) in toluene (200 mL), and nitrogen was bubbled through the mixture for 10 min. Then, copper iodide (1.04 g, 5.40 mmol) and 1,10 phenanthroline (1.96 g, 10.90 mmol) were added to the mixture and heated at reflux conditions (150° C.) and stirred for 48 h.

The mixture was washed with water (200 mL) and the aqueous layer was extracted with ethyl acetate (2×100 mL). The organic layers were combined, dried over sodium sulfate, filtered and concentrated under reduced pressure. The crude residue was purified via silica gel flash column chromatography using an 80 g silica cartridge and heptane:ethyl acetate 10-20% as solvent system in order to afford the desired compound, S-(3-((benzylthio)methyl)phenyl) ethanethioate (2.37 g, 76% yield).

### Step 3—Preparation of 1-Benzyl-2-(3-((benzylthio)methyl)phenyl)disulfane

A 1M solution of potassim hydroxide (1.68 mL, 1.68 mmol) was added dropwise to a solution of S-(3-((benzylthio)methyl)phenyl) ethanethioate (440 mg, 1.53 mmol) in methanol (5 mL) and tetrahydrofuran (2 mL), previously cooled to −40° C. with an acetonitrile/cardice bath, keeping the temperature below −35° C. The reaction mixture was stirred for 20 minutes and was then cooled to −70° C. with an acetone/cardice bath.

A solution of S-benzyl 4-methylbenzenesulfonothioate (638 mg, 2.29 mmol) in tetrahydrofuran (5 mL) was added dropwise. The resulting mixture was stirred for 20 min. at −70° C. and 1h at room temperature.

The mixture was neutralised with a saturated ammonium chloride aqueous solution and partitioned between ethyl acetate (50 mL) and more saturated ammonium chloride aqueous solution (40 mL). The aqueous layer was further extracted with ethyl acetate (50 mL). The organic layers were combined, dried over sodium sulfate, filtered and concentrated under reduced pressure. The crude residue was purified via silica gel flash column chromatography using a 120 g silica cartridge and heptane:ethyl acetate 1% as solvent system in order to afford the desired compound, 1-benzyl-2-(3-(benzylthio)phenyl)disulfane (0.52 g, 92% yield).

Step 4—Preparation of 1-Benzyl-2-(3-((benzylsulfinyl)methyl)phenyl)disulfane

3-Chloroperbenzoic acid (77%) (380 mg, 1.69 mmol) was added to a solution of 1-benzyl-2-(3-((benzylthio)methyl)phenyl)disulfane (520 mg, 1.41 mmol) in dichloromethane (20 mL), previously cooled to −70° C. using a cardice/acetone bath (keeping the temperature below −65° C. during the addition). The reaction mixture was stirred −70° C. for 10 min. and at room temperature for 1 h.

After that time, the mixture was diluted with dichloromethane (20 mL) and washed with an aqueous solution of sodium bicarbonate (40 mL). The aqueous layer was further extracted with dichloromethane (2×20 mL) and the organic layers were combined, dried over sodium sulfate, filtered and concentrated in vacuo in order to give a brown oil. The crude residue was purified via silica gel flash column chromatography using a 20 g silica cartridge and hexane:ethyl acetate 40% as solvent system in order to afford the desired compound, 1-benzyl-2-(3-(benzylsulfinyl)phenyl)disulfane (387 mg, 71% yield).

### Step 5—Preparation of 1-Benzyl-2-(3-((benzylsulfonyl)methyl)phenyl)disulfane

A solution of potassium permanganate (260 mg, 1.64 mmol) in acetone (15 mL) was added dropwise, over a period of 10 min., to a mixture of 1-benzyl-2-(3-((benzylsulfinyl)methyl)phenyl)disulfane (290 mg, 0.75 mmol) and magnesium sulfate (909 mg, 7.47 mmol) in acetone (20 mL), previously cooled down to −40° C. The internal temperature was kept below −35° C. during the addition. The reaction mixture was stirred at −40° C. for 20 min. and at −20° C. for 2 h. The mixture was filtered through a pad of celite and solvent was removed in vacuo. Further manganese III was removed by filtration through another pad of celite with more acetone. The filtrates were concentrated under reduced pressure in order to give a white solid. The crude material was purified via silica gel flash column chromatography using a 12 g silica cartridge and hexane:ethyl acetate 5-10%, hexane and hexane:dichloromethane (1:2) as solvent systems in order to afford the desired compound, 1-benzyl-2-(3-(benzylsulfonyl)phenyl)disulfane, as a white solid (32 mg, 11% yield).

### Example 2

### Method of Preparation of 1-Benzyl-2-(2-((benzylsulfinyl)methyl)phenyl)disulfane and 1-benzyl-2-(2-(benzylsulfonyl)phenyl)disulfane

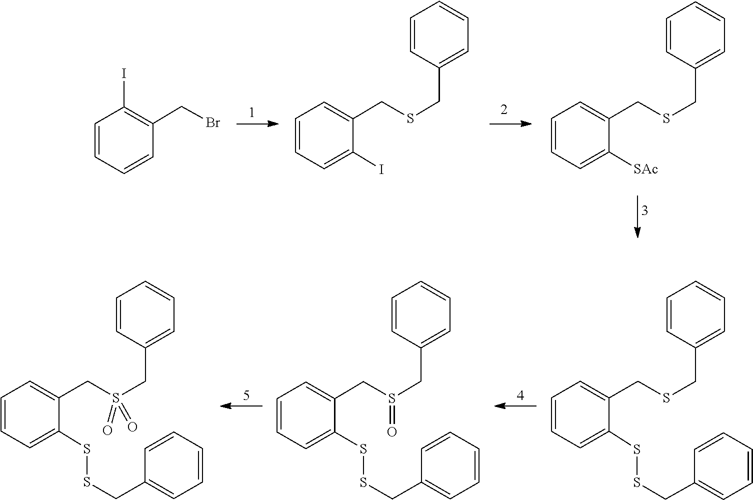

### Step 1—Preparation of Benzyl(2-iodobenzyl)sulfane

Benzyl mercaptan (2.0 mL, 16.80 mmol) was added dropwise to a suspension of sodium hydride (60% in mineral oil) (672 mg, 16.30 mmol) in tetrahydrofuran (100 mL), over a period of 10 minutes. The reaction mixture was stirred for a further 15 minutes at room temperature and a solution of 2-bromomethyl iodobenzene (5.00 g, 16.80 mmol) in tetrahydrofuran (20 mL) was added. The mixture was stirred at room temperature overnight.

The reaction mixture was quenched with a saturated solution of ammonium chloride (10 mL) and volatiles were removed in vacuo. The residue was partitioned between water (200 mL) and toluene (200 mL). The aqueous layer was further extracted with toluene (50 mL). The organic layers were combined, dried over sodium sulfate, filtered and concentrated under reduced pressure in order to give a pale brown oil. The crude residue was purified via silica gel flash column chromatography using a 120 g silica cartridge and heptane:dichloromethane 10% as solvent system in order to afford the desired compound, benzyl(2-iodobenzyl)sulfane, as a viscous colourless oil (5.30 g).

### Step 2—Preparation of S-(2-((Benzylthio)methyl)phenyl) ethanethioate

Potassium thioacetate (1.63 g, 14.30 mmol) was added to a solution of benzyl(2-iodobenzyl)sulfane (3.24 g, 9.52 mmol) in toluene (200 mL) and nitrogen was bubbled through the mixture for 10 minutes. Then, copper iodide (0.91 g, 4.76 mmol) and 1,10 phenanthroline (1.72 g, 9.52 mmol) were added to the mixture and it was heated to 100° C. (degassing was continued while heating to 100° C). The mixture was stirred at 100° C. for 48 h.

The mixture was cooled to room temperature and partitioned between water (250 mL) and ethyl acetate (100 mL). The aqueous layer was extracted with ethyl acetate (100 mL) and the organic layers were combined, dried over sodium sulfate, filtered and concentrated under reduced pressure. The crude residue was purified via silica gel flash column chromatography using a 80 g silica cartridge and heptane:dichloromethane 10-20% as solvent system in order to afford the desired compound, S-(2-((benzylthio)methyl)phenyl) ethanethioate (2.10 g).

### Step 3—Preparation of 1-benzyl-2-(2-((benzylthio)methyl)phenyl)disulfane

A 1M solution of potassium hydroxide (2.17 mL, 2.17 mmol) was added dropwise to a solution of S-(2-((benzylthio)methyl)phenyl) ethanethioate (418 mg, 1.45 mmol) in methanol (5 mL) and tetrahydrofuran (2 mL), previously cooled to −35° C. with an acetonitrile/cardice bath, keeping the temperature below −35° C. The reaction mixture was stirred for 20 minutes and then cooled to −70° C. with an acetone/cardice bath. A solution of S-benzyl 4-methylbenzenesulfonothioate (604 mg, 2.17 mmol) in tetrahydrofuran (5 mL) was added dropwise and the resulting mixture was stirred for 20 min. at −70° C. and 1 h at room temperature.

The mixture was neutralised with a saturated ammonium chloride aqueous solution (10 mL) and partitioned between ethyl acetate (40 mL) and more saturated ammonium chloride aqueous solution (20 mL). The aqueous layer was further extracted with ethyl acetate (2×20 mL). The organic layers were combined, dried over sodium sulfate, filtered and concentrated under reduced pressure. The crude residue was purified via silica gel flash column chromatography using a 12 g silica cartridge and heptane:ethyl acetate 2% as solvent system in order to afford the desired compound, 1-benzyl-2-(2-(benzylthio)phenyl)disulfane (320 mg).

### Step 4—Preparation of 1-Benzyl-2-(2-((benzylsulfinyl)methyl)phenyl)disulfane

3-Chloroperbenzoic acid (77%) (234 mg, 1.04 mmol) was added to a solution of 1-benzyl-2-(2-((benzylthio)methyl)phenyl)disulfane (320 mg, 0.87 mmol) in dichloromethane (20 mL) keeping the temperature below −65° C. The mixture was stirred for 10 minutes at −70° C. and 1 h at room temperature.

After that time, the mixture was washed with an aqueous solution of sodium bicarbonate and the aqueous layer was further extracted with dichloromethane (2×20 mL). The organic layers were combined, dried over sodium sulfate, filtered and concentrated in vacuo. The crude residue was purified via silica gel flash column chromatography using a 12 g silica cartridge and heptane:ethyl acetate 30-40% as solvent system in order to afford the desired compound, 1-benzyl-2-(2-(benzylsulfinyl)phenyl)disulfane, as a yellow solid (226 mg).

### Step 5—Preparation of 1-Benzyl-2-(2-((benzylsulfonyl)methyl)phenyl)disulfane

A solution of potassium permanganate (159 mg, 1.01 mmol) in acetone (15 mL) was added dropwise to a solution of 1-benzyl-2-(2-((benzylsulfinyl)methyl)phenyl)disulfane (176 mg, 0.46 mmol), previously cooled to −40° C. The reaction mixture was stirred for 5 h at −40° C. and 1 h at −20° C. before filtering it through a pad of celite.

Solvent was removed in vacuo and the crude material was purified via silica gel flash column chromatography using a 12 g silica cartridge and iso-hexane:ethyl acetate 10-15% as solvent system in order to afford the desired compound, 1-benzyl-2-(2-(benzylsulfonyl)phenyl)disulfane (53 mg, 29% yield).

### Example 3

### Method of preparation of 1-methyl-2-(3-((phenylsulfinyl)methyl)phenyl)disulfane and 1-methyl-2-(3-((phenylsulfonyl)methyl)phenyl)disulfane

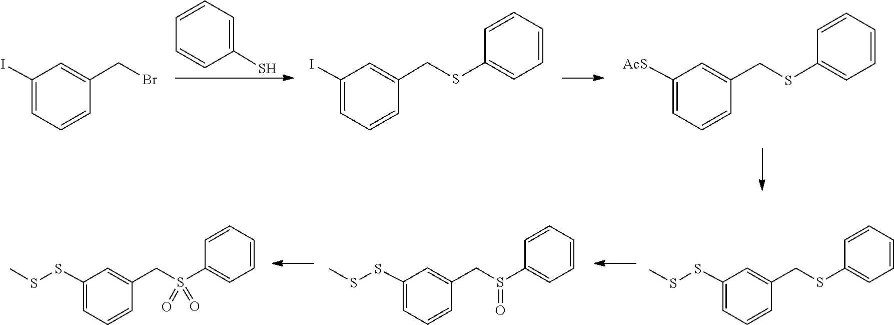

### Step 1—Preparation of 3-Iodobenzyl)(phenyl)sulfane

Thiophenol (0.50 mL, 4.90 mmol) was added dropwise to a mixture of sodium hydride (213 mg, 5.34 mmol) in tetrahydrofuran (10 mL), previously cooled to 5° C. The reaction mixture was stirred for 20 min. at 5° C. followed by the addition of a solution of 4-iodobenzyl bromide (1.20 g, 4.45 mmol) in tetrahydrofuran (10 mL) and the mixture was stirred for 1 h at room temperature.

The reaction mixture was quenched with a saturated solution of ammonium chloride (2 mL). The majority of the volatiles were removed in vacuo and the resulting oil and solid were partitioned between toluene (50 mL) and water (50 mL). The aqueous layer was further extracted with toluene (2×25 mL) and the organic layers were combined, dried over sodium sulfate, filtered and concentrated under reduced pressure in order to afford a pale yellow oil. The crude material was purified via silica gel flash column chromatography using a 40 g silica cartridge and heptane:dichloromethane 0-4% as solvent system in order to afford the desired product, (3-iodobenzyl)(phenyl)sulfane, as a colourless oil (1.22 g, 84% yield).

### Step 2—Preparation of S-(3-((Phenylthio)methyl)phenyl) ethanethioate

Potassium thioacetate (420 mg, 3.67 mmol) was added to a solution of (3-Iodobenzyl)(phenyl)sulfane (1.20 g, 3.67 mmol) in toluene (40 mL). The resulting mixture was stirred and degassed with nitrogen for 20 min. After that time, 1,10-Phenanthroline (661 mg, 3.67 mmol) and copper iodide (350 mg, 1.84 mmol) were added and the reaction mixture was heated to 110° C. (external temperature) and stirred under nitrogen for 60 h.

The mixture was cooled to room temperature, filtered through celite and washed with ethyl acetate (200 mL). The filtrates were washed with brine (100 mL) and the organic layer was dried over sodium sulfate, filtered and concentrated under reduced pressure. The crude material was purified via silica gel flash column chromatography using a 40 g silica cartridge and heptane:ethyl acetate 4% as solvent system in order to afford the desired product, S-(3-((phenylthio)methyl)phenyl) ethanethioate, as an orange viscous oil (690 mg, 69% yield).

### Step 3—Preparation of 1-Methyl-2-(3-((phenylthio)methyl)phenyl)disulfane

A three neck round-bottom flask (equipped with a thermometer, septum and a calcium chloride guard tube) was charged with S-(3-((phenylthio)methyl)phenyl) ethanethioate (680 mg, 2.48 mmol) and methanol (25 mL). The mixture was cooled to −35° C. and a 1M solution of potassium hydroxide in methanol (3.2 mL, 3.20 mmol) was added dropwise over a period of 5 min. The reaction mixture was stirred for 30 min. keeping the internal temperature around −35° C. After that time, the mixture was cooled further to −65° C. and a solution of methylthiotosylate (652 mg, 3.23 mmol) in methanol (25 mL) was added dropwise over a period of 20 min. keeping the temperature around −60° C. The reaction mixture was stirred for 3 h whilst allowing the temperature to rise to 0° C.

The reaction mixture was poured into a saturated sodium bicarbonate aqueous solution (200 mL) and extracted with tent-butylmethyl ether (3×80 mL). The organic layers were combined, washed with brine (100 mL), dried over sodium sulfate, filtered and concentrated in vacuo. The crude material was purified via silica gel flash column chromatography using a 20 g silica cartridge and heptane:ethyl acetate 2% as solvent system in order to afford the desired product, 1-methyl-2-(3-((phenylthio)methyl)phenyl)disulfane, as a yellow oil (630 mg, 91% yield).

### Step 4—Preparation of 1-Methyl-2-(3-((phenylsulfinyl)methyl)phenyl)disulfane

3-Chloroperbenzoic acid (513 mg, 2.23 mmol) was added in one portion to a solution of 1-methyl-2-(3-((phenylthio)methyl)phenyl)disulfane (620 mg, 2.23 mmol) in dichloromethane (50 mL), previously cooled to −65° C. The reaction mixture was stirred for 3 h whilst the temperature was allowed to warm to 0° C. As LCMS analysis showed the reaction was not complete, more 3-chloroperbenzoic acid (51 mg, 0.22 mmol) was added and the reaction mixture was stirred for a further 2 h whilst the temperature was allowed to warm from 0° C. to room temperature.

The mixture was diluted with dichloromethane (200 mL) and washed with a saturated sodium bicarbonate aqueous solution (2×100 mL) and water (100 mL). The organic layer was dried over sodium sulfate, filtered and concentrated in vacuo. The crude material was purified via silica gel flash column chromatography using a 40 g silica cartridge and heptane:ethyl acetate 40% as solvent system in order to afford the desired product, 1-methyl-2-(3-((phenylsulfinyl)methyl)phenyl)disulfane, as a colourless, viscous oil (500 mg, 76% yield).

### Step 5—Preparation of 1-Methyl-2-(3-((phenylsulfonyl)methyl)phenyl)disulfane

A mixture of 1-methyl-2-(3-((phenylsulfinyl)methyl)phenyl)disulfane (340 mg, 1.16 mmol) and magnesium sulfate (702 mg, 5.83 mmol) in acetone (20 mL) was stirred and cooled to −20° C. A solution of potassium permanganate (194 mg, 1.23 mmol) in acetone (35 mL) was added dropwise over a period of 1 h, keeping the temperature below 0° C. The reaction mixture was stirred for 4.5 h at 5° C.

The mixture was filtered through celite and washed with acetone (150 mL). The filtrates were concentrated in vacuo and the crude material was purified via silica gel flash column chromatography using a 20 g silica cartridge and heptane:ethyl acetate 20% as solvent system in order to afford the desired product, 1-methyl-2-(3-((phenylsulfonyl)methyl)phenyl)disulfane, as a colourless, viscous oil/gum (290 mg, 80% yield).

### Example 4

### Minimum Biofilm Inhibition Concentration (MBIC) of 1-(phenylmethanesulfonylmethyl)-3-(prop-2-en-1-yldisulfanyl)benzene [Compound A]

The inhibitory activity of the following compound of formula (I) on Staphylococcus aureus and Pseudomonas aeruginosa was compared to ajoene, cysteamine, HDMF, gallium nitrate, and (Z-)-4-Bromo-5-(bromomethylene)-2(5H)-furanone (furanone C-30).

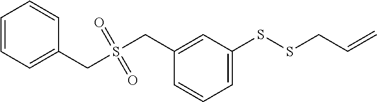 [SMILES: C=CCSSc1cccc(CS(=O)(=O)Cc2ccccc2)c1]

The structures of cysteamine, HDMF, gallium nitrate, and C-30 are shown below.

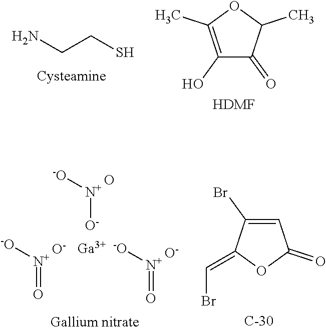

Compounds were assayed for their ability to inhibit S. aureus or P. aeruginosa biofilm formation as follows.

All compounds were prepared as a 30 mM stock in DMSO and diluted in tryptic soy broth (TSB) to 48 μM (S. aureus) or 144 μM (P. aeruginosa). The compounds were further serially diluted two-fold from 48 μM to 0.75 μM (S. aureus) or 144 μM to 2.3 μM (P. aeruginosa) and added to a 96-well plate at 100 μl per well. Overnight cultures of bacteria were diluted to an OD₆₀₀=0.07 in fresh TSB and 100 μl of the diluted bacterial culture was added to the compound containing wells. The bacteria were incubated in the presence of the compounds overnight at 37° C. After incubation, the planktonic bacteria were removed by three washes using H₂O and the remaining biofilm was stained using crystal violet and quantified photometrically at A₅₇₀. Absorbances corresponding to biofilm mass were plotted versus compound concentration and IC₅₀ values were calculated. Reference: O'Toole G A, Microtiter Dish Biofilm Formation Assay J. Vis. Exp (2011).

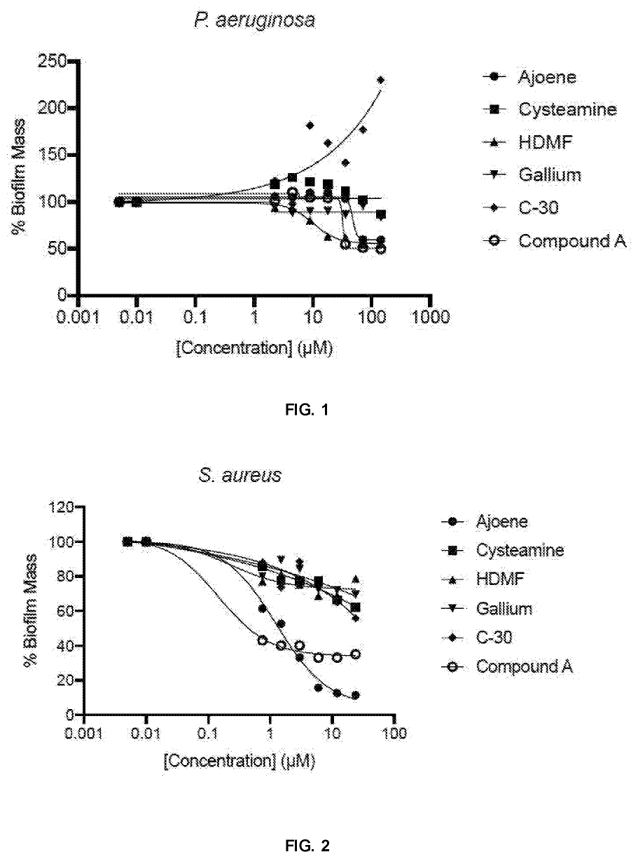

The results are shown in FIGS. 1 and 2. It was surprisingly found that compounds of formula (I) had a lower IC50 than all of ajoene, cysteamine, HDMF, gallium nitrate, and C-30 for both Staphylococcus aureus and Pseudomonas aeruginosa.

### Example 5

### Inhibition of Respiration/Viability of P. aeuruainosa Biofilms Drown in Artificial Sputum Medium (ASM) by 1-(methyldisulfanyl)-3-(phenylmethanesulfonylmethyl)benzene (Compound B) and Tobramycin

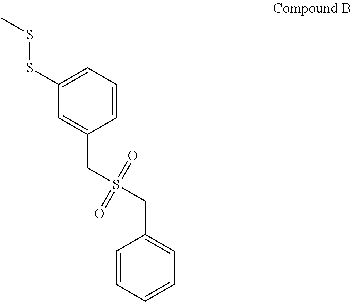 [SMILES: CSSc1cccc(CS(=O)(=O)Cc2ccccc2)c1]

Compound B was tested for its efficacy to inhibit P. aeruginosa biofilms in artificial sputum medium (ASM) as follows. Overnight cultures of P. aeruginosa were re-seeded to an OD595=0.07 and further diluted 1:100 in ASM and grown for 24 hours at 37° C. to allow for biofilm formation. Compound B was prepared as 30 mM stock in 100% DMSO and established biofilms were treated with the compound at increasing concentrations or left untreated for 5 hours at 37° C. All samples were then treated with 10 μg/ml tobramycin for 16 hours at 37° C. The artificial sputum was liquified using sputasol. The respiratory potential of the biofilms was quantified using a tetrazolium chloride (2,3,5-triphenyl tetrazolium chloride (TTZ)) viability assay.

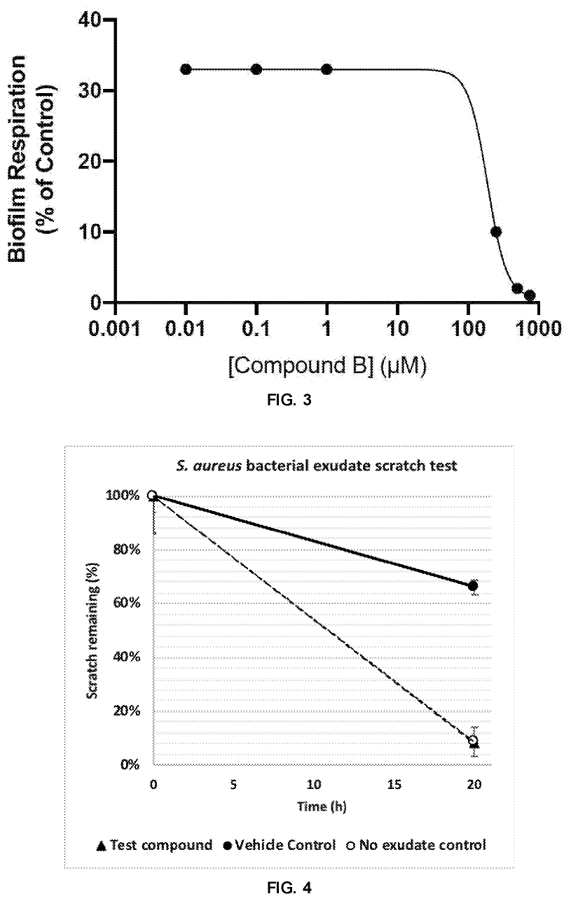

The results are shown in FIG. 3. It was surprisingly found that compound B enhances the efficacy of tobramycin to inhibit the respiration/viability of P. aeruginosa biofilms grown in artificial sputum medium.

### Example 6

### Minimum Biofilm Inhibition Concentration (MBIC) Assay in Reference to Ajoene Control

MBIC assays of the compounds are carried out alongside ajoene for reference and standardization. The IC50 of ajoene against S. aureus and P.aeruginosa is well-defined and reproducible allowing ajoene to serve as an effective control for the experiment. As such, the IC50 calculated for the tested compounds are compared to the IC50 of ajoene in every experiment and are therefore represented as % of ajoene.

If in a given experiment a compound had an IC50 at 100% of ajoene, this compound would be equally as effective as ajoene. If a compound has an IC50<100% of ajoene then this compound is more effective than ajoene. If a compound has an IC50>100% of ajoene then this compound is less effective than ajoene.

Table 1 below shows MBIC results of representative compounds having the structure of Formula (I).

TABLE 1

| | | S. |
|---|---|---|
| | P. | aureus |
| | aeruginosa | MBIC: |
| | MBIC: % of | % of |
| | Ajoene | Ajoene |
| | positive | positive |
| | control | control |
| Structure | IC50 | IC50 |
| 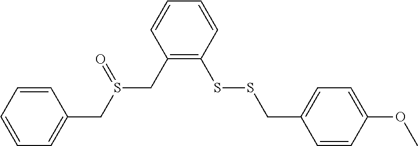 [SMILES: COc1ccc(CSSc2ccccc2CS(=O)Cc2ccccc2)cc1] | 58 | 4 |
| 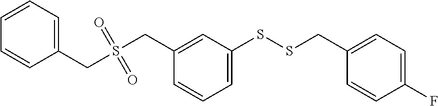 [SMILES: O=S(=O)(Cc1ccccc1)Cc1cccc(SSCc2ccc(F)cc2)c1] | 35 | 69 |
| 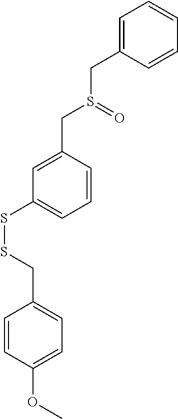 [SMILES: COc1ccc(CSSc2cccc(CS(=O)Cc3ccccc3)c2)cc1] | 20 | 43 |
| 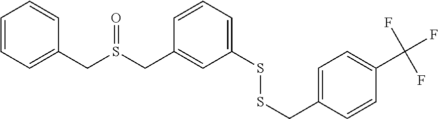 [SMILES: O=S(Cc1ccccc1)Cc1cccc(SSCc2ccc(C(F)(F)F)cc2)c1] | 32 | 34 |
| 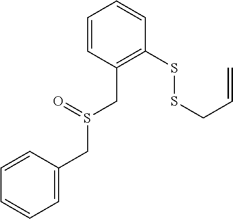 [SMILES: C=CCSSc1ccccc1CS(=O)Cc1ccccc1] | 76 | 89 |
| 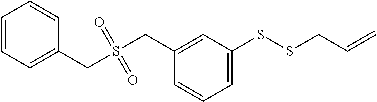 [SMILES: C=CCSSc1cccc(CS(=O)(=O)Cc2ccccc2)c1] | 82 | 5 |
| 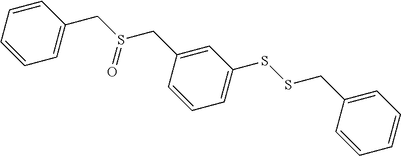 [SMILES: O=S(Cc1ccccc1)Cc1cccc(SSCc2ccccc2)c1] | 21 | 62 |
| 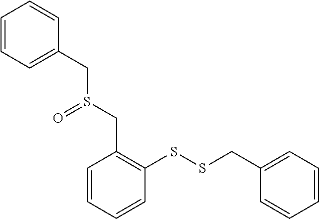 [SMILES: O=S(Cc1ccccc1)Cc1ccccc1SSCc1ccccc1] | 39.9 | 89 |
| 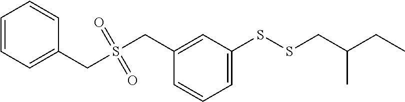 [SMILES: CCC(C)CSSc1cccc(CS(=O)(=O)Cc2ccccc2)c1] | 94 | 11 |
| 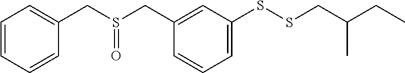 [SMILES: CCC(C)CSSc1cccc(CS(=O)Cc2ccccc2)c1] | 15 | 22.5 |
| 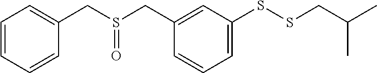 [SMILES: CC(C)CSSc1cccc(CS(=O)Cc2ccccc2)c1] | 32 | 90 |
| 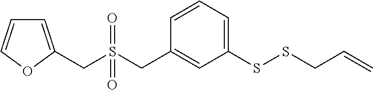 [SMILES: C=CCSSc1cccc(CS(=O)(=O)Cc2ccco2)c1] | 100 | 22 |

### Example 7

### Effect of Prophylactic Treatment on Bacterial Exudate Scratch Test (BEST)

The experiment aim was to demonstrate the effect of a test compound on secreted virulence factors (VF) via survival and proliferation of a scratched cell HaCaT monolayer using a non-lethal dose of the test compound (n=4).

The test compound was

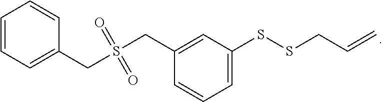 [SMILES: C=CCSSc1cccc(CS(=O)(=O)Cc2ccccc2)c1]

Bacterial cultures (S. aureus and P. aeruginosa) were grown prophylactically with test compounds for 24 hours and exudates were collected.

Microorganisms were prepared by growing in tryptone soy broth (TSB) at 37° C., 150 rpm for 16 hours.

The exudates were obtained by first placing in a 24-well plate, 1 mL of each compound treated media and vehicle control media in triplicate. 1 mL of untreated TSB was then added to 3 wells for an untreated control and 2 mL TSB was added to 3 wells for sterility control. Except for the sterility control wells, 1 mL of bacterial suspension was added to each well and mixed well. The plate was then incubated in a humidified incubator at 37° C. for 24 hours at 75 rpm. Following the appropriate incubation time, the entire contents of the well were removed and transferred into a sterile centrifuge tube. The tube was then centrifuged at 10,000 rpm for 10 minutes at 4° C. to pellet the biomass. The supernatant was filtered using a Luer Lock 0.2 μM filter into another sterile tube. The exudate was aliquoted into 500 μl aliquots in 0.5 mL PCR tubes. The exudates were diluted to 1:20 in phenol red free medial alpha (MEM).

HaCaT cells were seeded in a 96-well plate at a density of 5×10⁴ cells per well and incubated overnight at 37° C., 5% CO₂ and 95% humidity. The HaCaT cells were washed with pre-warmed PBS and the monolayers mechanically injured by introducing a vertical cell-free area in each individual well and washed again.

100 μl of exudate was then contacted with the monolayer. All conditions were tested with an N of 4 replicates.

The plates were incubated for 20 hours at 37° C., 5% CO₂ and images were taken at the time points as required. Scratch ‘size’ was analysed using ImageJ software.

The rate of cellular proliferation of the HaCaT cells is calculated at 20 hours as a percentage of the initial scratch size at 0 hours.

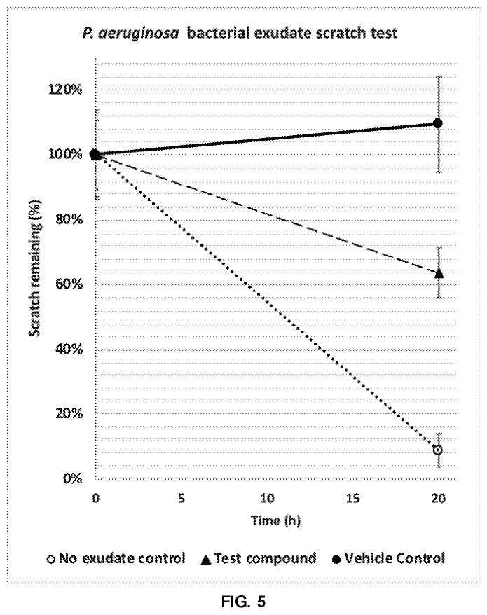

The results are shown in FIGS. 4 and 5.

It was found that prophylactic (pre-biofilm) treatment of S. aureus with the test compound (125 pM) completely rescues the inhibition of scratch closure in HaCaT cells compared to bacterial exudates obtained by pre-treatment with vehicle. HaCaTs exposed to the bacterial exudates obtained by pre-treatment with the test compound have almost complete closure of the scratch area at 20 hours compared to the 66% remaining on exposure to bacterial exudates obtained by pre-treatment with vehicle.

It was found that prophylactic (pre-biofilm) treatment of P. aeruginosa with the test compound (250 μM) reduces the inhibition of scratch closure in HaCaT cells compared to bacterial exudates obtained by pre-treatment with vehicle. HaCaTs exposed to bacterial exudates obtained by pre-treatment with vehicle show an increased scratch area to 109% at 20 hours compared to a reduction of scratch area to 9% for HaCaTs not exposed to bacterial exudate. Bacterial exudate obtained by pre-treatment with the test compound reduces the scratch area to 64% at 20 hours.

The foregoing broadly describes certain embodiments of the present invention without limitation. Variations and modifications as will be readily apparent to those skilled in the art are intended to be within the scope of the present invention as defined in and by the appended claims.

1. A compound of formula (I), 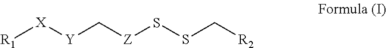
wherein:
R₁ is phenyl, substituted phenyl, cycloalkyl, substituted cycloalkyl, heterocyclyl, substituted heterocyclyl, or alkyl;
X is (CH₂)ₙ;
n is 0 to 10;
Y is sulphuryl or sulphinyl;
Z is phenyl or substituted phenyl;
R₂ is hydrogen, phenyl, substituted phenyl, alkyl, alkenyl, heterocyclyl, substituted heterocyclyl, —CH(CO₂R₃)(NHCO₂R₄), —CH(CO₂H)(NH₂), or —CH(CO₂R₅)(R₆); and
R₃, R₄, R₅, and R₆ are alkyl;
or a pharinaceutically acceptable salt, ester, or prodrug thereof;
excluding the compound:

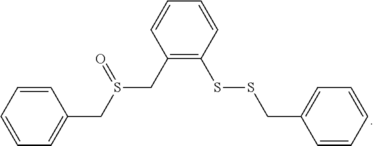 [SMILES: O=S(Cc1ccccc1)Cc1ccccc1SSCc1ccccc1]

2. The compound of claim 1, wherein the compound is according to formula (IA) or (IB),

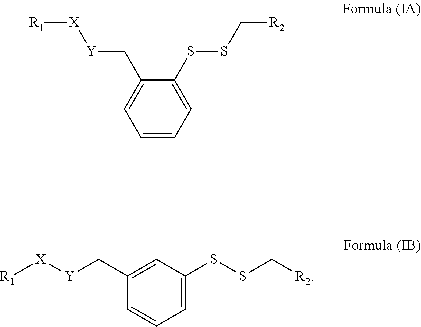

3. The compound of claim 1, wherein R₁ is phenyl, substituted phenyl, or alkyl,

4. The compound of claim 1, wherein R₁ is phenyl.

5. The compound of claim 1, wherein R₂ is hydronen, phenyl, substituted, phenyl, alkenyl, heterocyclyl, or substituted heterocyclyl.

6. The compound of claim 1, wherein R₂ is hydrogen, alkyl, or alkenyl.

7. The compound of claim 1, wherein n is 0 or 1.

8. The compound of claim 1, wherein Y is sulphuryl.

9. The compound of claim 1, wherein Z is phenyl.

10. The compound of claim 1, wherein R₂ is ethenyl.

11. The compound of claim 1, wherein R₂ is substituted phenyl, substituted with one of alkoxy, halogen, sulphonyl, or haloalkyl.

12. The compound of claim 1, wherein R₂ is substituted phenyl, substituted with one of methoxy, fluoro, methyl sulphonyl, or trifluoromethyl.

13. The compound of claim 1, wherein R₃ is substituted phenyl, substituted at a para position of a phenyl ring.

14. A pharmaceutical composition comprising a compound of claim 1 and a pharmaceutically acceptable excipient and/or carrier and/or diluent.

15. A compound or pharmaceutical composition of formula (I) or a pharmaceutically acceptable salt, ester, or prodrug thereof, for use in a therapeutic method for treating a microbial infection and/or for treating inflammation and/or for reducing the formation of blood clots, 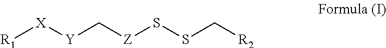
wherein:
R₁ is phenyl, substituted phenyl, cycloalkyl, substituted cycloalkyl, heterocyclyl, substituted beterocyclyl, or alkyl;
X is (CH₂)ₘ;
n is 0 to 10;
Y is sulphuryl or sulphinyl;
Z is phenyl or substituted phenyl;
R₂ is hydrogen, phenyl, substituted phenyl, alkyl, alkenyl, heterocyclyl, substituted heterocyclyl, —CH(CO₂R₃)(NHCO₂R₄), —CH(CO₂H)(NH₂), or —CH(CO₂R₅)(R₆); and
R₃, R₄, R₅, and R₆ are alkyl.

16. (canceled)

17. A therapeutic method for treating a microbial infection and/or for treating inflammation and/or for reducing the formation of blood clots, wherein the method comprises administering a compound according to claim 15, or a pharmaceutical composition or a pharmaceutically acceptable salt, ester, or prodrug thereof to a subject.

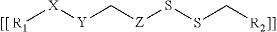

18. A non-therapeutic use of the compound of claim 15, or a pharmaceutically acceptable salt, ester, or prodrug thereof, as an antimicrobial agent and/or as an anti-inflammatory agent and/or as an anti-thrombotic agent.

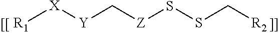

19. (canceled)

20. A method for making a compound of formula (I), wherein the method proceeds via steps 1 to 4 or steps 1 to 5 of reaction scheme (IA) or (IB): 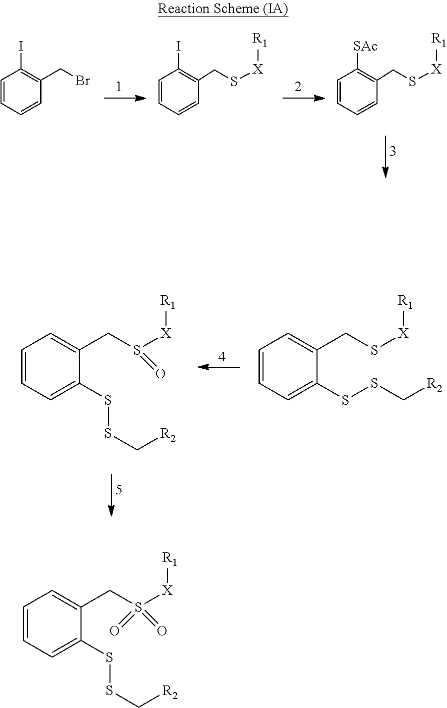 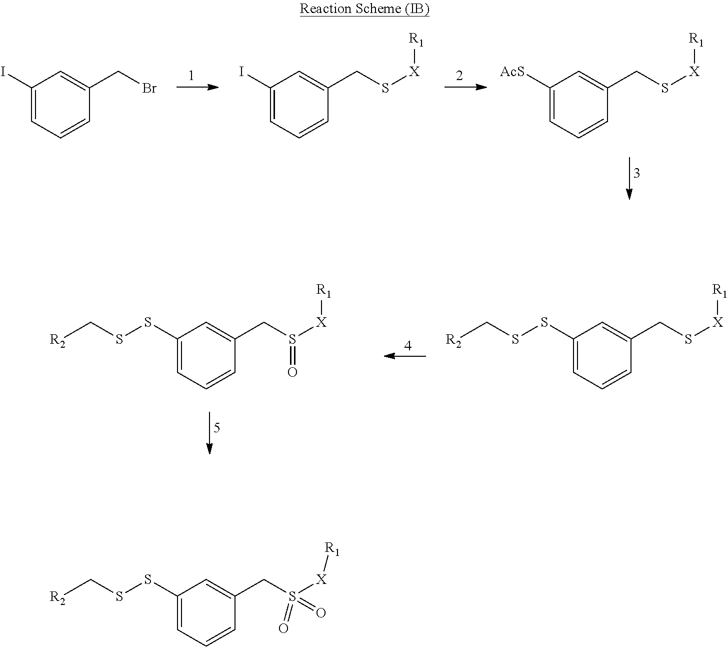
wherein:
R₁ is phenyl, substituted phenyl, cycloalkyl, substituted cycloalkyl, heterocyclyl, substituted heterocyclyl, or alkyl;
X is (CH₂)ₙ;
n is 0 to 10;
Y is sulphuryl or sulphinyl;
Z is phenyl or substituted phenyl;
R₂ is hydrogen, phenyl, substituted phenyl, alkyl, alkenyl, heterocyclyl, substituted heterocyclyl, —CH(CO₂R₃)(NHCO₂R₄), —CH(CO₂H)(NH₂), or —CH(CO₂R₅)(R₆); and
R₃, R₄, R₅, and R₆ are alkyl.

21. The compound of claim 2, wherein R₁ is phenyl, substituted phenyl, or alkyl, and n is 0 or 1.

22. The compound of claim 21, wherein Y is sulphuryl, and R₂ is ethenyl.
